SEQUENCE LISTING (1 sequence)

SEQ ID NO: 1 - protein, 269 aa, synthetic construct [sequence omitted]

(19) United States
(12) Patent Application Publication
(10) Pub. No.: US 2025/0011382 A1
(43) Pub. Date: Jan. 9, 2025

(54) METHOD FOR THE MANUFACTURE OF BIOPHARMACEUTICALS

(71) Applicant: RICHTER GEDEON NYRT., Budapest (HU)

(72) Inventors: Jan Herzog, Hamburg (DE); Patrick Wegener, Bornhöved (DE)

(21) Appl. No.: 18/890,829

(22) Filed: Sep. 20, 2024

Related U.S. Application Data
(63) Continuation of application No. PCT/EP2023/057635, filed on Mar. 24, 2023.

(30) Foreign Application Priority Data
Mar. 24, 2022 (EP) 22164126.9

(51) Int. Cl. C07K 14/52; A61K 47/22; A61K 47/26; C12N 1/20; C12P 21/02; C12R 1/19

(52) U.S. Cl. CPC C07K 14/524; A61K 47/22; A61K 47/26; C12N 1/20; C12P 21/02; C07K 2319/30; C12R 2001/19

(57) ABSTRACT

Provided is a thermo-regulated process for the recombinant production of an Fc-peptide fusion protein in prokaryotic host cells comprising a single mild temperature shift in the induction phase.

### CROSS-REFERENCE

This application is a continuation of International Application No. PCT/EP2023/057635, filed Mar. 24, 2023, which claims priority to European Patent Application No. 22164126.9, filed Mar. 24, 2022, the content of which is incorporated herein by reference in its entirety.

### BRIEF DESCRIPTION OF THE SEQUENCE LISTING

The instant application contains a Sequence Listing which has been submitted electronically in XML format and is hereby incorporated by reference in its entirety. The XML copy, created on Mar. 16, 2023, is named sequence listing.xml and is 2,392 bytes in size.

### FIELD OF THE INVENTION

The present invention relates to a process for the recombinant production of an Fc-peptide fusion protein in prokaryotic host cells. Furthermore, the present invention relates to pharmaceutical compositions comprising an Fc-peptide fusion protein obtained by said process and to methods of manufacturing such pharmaceutical compositions.

### BACKGROUND OF THE INVENTION

Peptides can be synthetically made with high purity and can be designed to modulate various biological processes with high specificity. Because of their small size and hence good tissue penetration, the use of peptides as pharmacological agents is an attractive approach. However, the therapeutic use of peptides is limited, as many biologically active peptides have a limited half-life due to their rapid renal clearance, which limits their exposure and pharmacological effects in the target tissue. In contrast, immunoglobulin G (IgG) exhibits a prolonged half-life due to its large size and due to the pH-dependent binding of the neonatal Fc-receptor (FcRn) that salvages the IgG from degradation (Pyzik et al., J. Immunol. 194 (2015), 4595-4603).

Fc-peptide fusion proteins, or “peptibodies” combine the biologic activity of peptides with the stability of monoclonal antibodies. The active high-affinity peptide is fused in frame to the Fc region of an IgG (Shimamoto et al., mAbs 4 (2012), 586-591). The integration of the Fc domain of an IgG leads to a prolongation of the circulation half-life through FcRn protection.

When it comes to producing recombinant proteins in high quantities and at low production costs, E. coli based expression systems have been the first choice for decades given the availability of a variety of well characterized strong inducible promoters, including those derived from gram-positive bacteria and bacteriophages (Makrides, Microbiological Reviews 60 (1996). 512-538). Since then. E. coli based expression systems have been subject to optimization for the production of recombinant proteins.

Hence, there is a constant need for providing robust and scalable manufacturing processes for recombinant proteins such as Fc-peptide fusion proteins with optimized process features to maximize protein yields within reasonable process times.

### SUMMARY OF THE INVENTION

The present invention generally relates to a process for the recombinant production of an Fc-peptide fusion protein in prokaryotic host cells which comprises a batch phase, a feeding phase, i.e. a fed-batch phase before induction, and an induction phase, also called expression phase.

Prokaryotic expression systems, and especially E. coli based expression systems are commonly based on the use of a strong inducible promoter and the addition of an inducer molecule such as IPTG and antibiotics, the depletion or addition of a nutrient, or a shift in a physical or physicochemical factor, such as temperature and pH. However, each of these options bears different disadvantages which may complicate the bioprocess and increase its cost (Valdez-Cruz et al., Microb Cell Fact. 9:18 (2010), doi: 10.1186/1475-2859-9-18).

In accordance with the present invention, based on a thermoregulated expression system a new cost efficient production process could be established which avoids a high induction temperature. The present invention is based on experiments using a temperature inducible expression system comprising the strong major leftward (pL) and/or rightward (pR) phage lambda promoters which are regulated by the mutant thermolabile cI857 repressor of bacteriophage lambda (Villaverde et al., Appl. Environ. Microbiol. 59 (1993), 3485-3487). Gene expression is inhibited at cultivation temperatures below 37° C.′, whereas transcription by the host RNA polymerase ensues upon inactivation of the mutant thermolabile cI857 repressor by increasing the temperature over 37° C. and usually up to 42° C. in order to reach a high expression level and thus, a high product concentration. In accordance with the present invention, it could be shown that a single mild temperature increase from about 30° C. to only about 39° C. leads to the efficient production of Fc-peptide fusion proteins which reduces the energy required to heat up the bioreactor system; see Example 1. Based on the experiments performed in accordance with the present invention, a scalable fed-batch fermentation process could be established for the production of any kind of Fc-peptide fusion protein, exemplified with romiplostim which was produced at a yield of 8.0 g/L. A flowchart of the established two-stage fermentation process including the applied control loops and set points is shown in FIG. 1. Besides the reduction of costs, the lower temperature rise is also beneficial for the production peptides and Fc-fusion proteins thereof which are prone to irreversible denaturation and chemical modification which happen to take place at about 42° C.

Therefore, the present invention relates to [1] a process suitable for the large scale recombinant production of an Fe-peptide fusion protein in prokaryotic host cells, wherein the process comprises a batch phase, a feeding phase before induction (pre-induction fed batch phase), and a temperature induction phase (expression phase), characterized by a) culturing the recombinant prokaryotic host cells comprising a nucleic acid encoding the Fc-peptide fusion protein and which is operably linked to a temperature inducible promoter during the batch phase and the feeding phase at a first cultivation temperature of about 27° C. to about 35° C. in a culture medium; b) starting the temperature induction phase at an OD₆₀₀ of >30 through a temperature shift towards a second cultivation temperature of about 38° C. to about 40° C. thereby inducing the expression of the Fc-peptide fusion protein; c) and maintaining the second cultivation temperature for at least 4 hours. The process of the present invention is especially suitable and useful for the large scale recombinant production of Fc-peptide fusion proteins and preferably performed in a bioreactor. In addition, though the process of the present invention has been illustrated by way of example with an Fc-peptide fusion protein and the following embodiments are described for the sake of clarity for an Fc-peptide fusion protein too, it is to be understood that the present invention extends to the use of the process for the recombinant production of any kind of protein which is amenable to the recombinant production in prokaryotic host cells such as E. coli.

Accordingly, the following embodiments of the process of the present invention, though described for Fc-peptide fusion proteins are applicable to the production of proteins, in particular therapeutic proteins.

[2] The process according to [1], which further comprises a step d) of harvesting the Fc-peptide fusion protein from the prokaryotic host cells or from the cell culture medium.

[3] The process according to [1] or [2], wherein the prokaryotic host cells are Escherichia coli cells.

[4] The process according to [3], wherein the Escherichia coli cells are Escherichia coli BL21 cells.

[5] The process according to any one of [1] to [4], wherein the temperature inducible promoter is the lambda PR promoter, which is regulated by a temperature sensitive repressor, preferably the lambda cI857 repressor.

[6] The process according to any one of [1] to [5], wherein culturing the recombinant prokaryotic host cells during the batch phase and the feeding phase according to step a) occurs at a first cultivation temperature of 30° C. #1° C.

[7] The process according to any one of [1] to [6], wherein culturing the recombinant prokaryotic host cells according to step a) occurs in a culture medium in the presence of glycerol as carbon source.

[8] The process according to any one of [1] to [7], wherein the medium used in the feeding phase does not comprise methionine.

[9] The process according to any one of [1] to [7], wherein the medium used in the batch phase and/or the feeding phase does not comprise an antibiotic, preferably does not comprise kanamycin.

[10] The process according to any one of [1] to [6], wherein the medium used in the feeding phase does not comprise methionine and wherein the medium used in the batch phase and the feeding phase does not comprise an antibiotic, preferably does not comprise kanamycin.

[11] The process according to any one of [1] to [10], wherein culturing the recombinant prokaryotic host cells according to step a) occurs at a growth rate of 0.05 to 0.3 doublings per hour, preferably of 0.1 doublings per hour.

[12] The process according to any one of [1] to [11], wherein starting the temperature induction phase according to step b) occurs at an OD₆₀₀ of 80±5, preferably of 82.5±2.5.

[13] The process according to any one of [1] to [12], wherein the second temperature is 39° C.

[14] The process according to any one of [1] to [13], wherein shifting the first cultivation temperature towards the second temperature occurs through ramping the temperature at a heating rate between 1° C./min and 6° C./min, preferably between 1° C./min and 4° C./min, more preferably of 3.6° C./min for a 10 L bioreactor.

[15] The process according to any one of [1] to [14], wherein the first cultivation temperature is 30° C. and the second cultivation temperature is 39° C.

[16] The process according to any one of [1] to [15], wherein the second temperature is maintained in step c) for 5 to 10 hours, preferably for 6 to 8 hours, more preferably for 8 hrs.

[17] The process according to any one of [1] to [16], wherein a dissolved oxygen (DO) concentration is maintained at above 8%, preferably at above 10%, most preferably at 30%.

[18] The process according to [17], wherein the DO concentration is maintained through control of bioreactor pressure, preferably by increasing the bioreactor pressure from 0.0 to 0.8 bar(g).

[19] The process according to any one of [1] to [18], wherein the Fe-peptide fusion protein is expressed in insoluble or limited soluble form and is accumulated in inclusion bodies.

[20] The process according to any one of [2] to [19], wherein process step d) further comprises a step of isolation of the Fc-peptide fusion protein containing inclusion bodies by a sedimentation step, preferably by a centrifugation step.

[21] The process according to any one of [2] to [20], wherein process step d) further comprises a step of refolding the Fc-peptide fusion protein.

[22] The process according to any one of [2] to [21], wherein process step d) further comprises a step of purifying the Fc-peptide fusion protein.

[23] The process according to [22], wherein the purification of the Fc-peptide fusion protein is performed by a method comprising the following steps in the following order:
a) performing an affinity capture chromatography in bind-elute mode;
b) performing a mixed-mode chromatography in bind-elute mode:
c) performing a cation exchange chromatography in bind-elute mode; and
d) performing an ultrafiltration/diafiltration;
wherein the chromatographic steps a), b), and c) are performed optionally with one or more washing steps.

[24] The process according to any one of [2] to [23], wherein harvesting/recovering the Fc-fusion peptide from the cell culture medium according to step d) comprises centrifugation of the cell culture medium and separation of the cell pellet for further downstream processing, optionally wherein the cell pellet is stored at −20° C.

[25] The process according to any of [1] to [24], wherein the Fc-peptide fusion protein is a monomer, a dimer, or a polymer, preferably a dimer.

[26] The process according to any of [1] to [25], wherein the Fc-peptide fusion protein comprises a native or synthetic peptide.

[27] The process according to any of [1] to [26], wherein the Fc-peptide fusion protein is a receptor agonist, preferably wherein the receptor agonist is a thrombopoietin mimetic.

[28] The process of any of [1] to [27], wherein the thrombopoietin mimetic is romiplostim.

Fc-peptide fusion proteins, also called peptibodies are valuable pharmaceuticals that can be used for the treatment of various diseases. Romiplostim, a thrombopoietin (TPO) analog fused to an IgG1, was the first peptibody approved by both EMA and FDA, followed by dulaglutide. a glucagon-like peptide-1 (GLP-1) fused to an IgG4. The efficacy and safety of both peptibodies favored the investment of researchers to explore this strategy for the development of new therapeutic molecules. Thus, a considerable number of different peptibodies are currently in preclinical and clinical studies.

Accordingly, the present invention also relates to a method of manufacturing a pharmaceutical composition comprising an Fc-peptide fusion protein, wherein the method comprises the steps of the process of any one of [1] to [28], and formulating the Fc-peptide fusion protein with a pharmaceutically acceptable carrier or buffer, wherein the method preferably comprises any one of the following embodiments.

[30] The method of [29], which further comprises filling the pharmaceutical composition in a pharmaceutical container.

[31] The method of [29] or [30], wherein the method comprises lyophilizing the pharmaceutical composition.

[32] The method of [29] to [31], wherein the Fc-peptide fusion protein is formulated with a buffer, and at least one further compound selected from a detergent, a stabilizer, a salt, a sugar, and a polyol.

[33] The method of any one of [29] to [31], wherein the Fc-peptide fusion protein is romiplostim and is formulated with L-histidine, mannitol, polysorbate 20, sucrose and HCL.

In addition, the present invention relates to the pharmaceutical composition obtained by the method of any one of [29] to [33].

The pharmaceutical composition of the present invention may be further characterized by the following embodiments.

[35] The pharmaceutical composition of [34], which is a sterile, preservative-free, white powder contained in a single-dose vial.

[36] The pharmaceutical composition of [35], wherein the vial comprises
a) 230 mcg romiplostim, 0.7 mg L-histidine, 18 mg mannitol, 0.02 mg polysorbate 20.9 mg sucrose, and sufficient HCL to adjust the pH to a target of 5.0;
b) 375 mcg romiplostim, 1.2 mg L-histidine, 30 mg mannitol, 0.03 mg polysorbate 20.15 mg sucrose, and sufficient HCl to adjust the pH to a target of 5.0; or
c) 625 mcg romiplostim, 1.9 mg L-histidine, 50 mg mannitol, 0.05 mg polysorbate 20.25 mg sucrose, and sufficient HCl to adjust the pH to a target of 5.0.

The present invention also relates to the [37] use of a recombinant prokaryotic host cell comprising a nucleic acid encoding an Fc-peptide fusion protein and which is operably linked to a temperature inducible promoter or a vector comprising the nucleic acid encoding an Fc peptide fusion protein and which is operably linked to a temperature inducible promoter in the process of any one of [1] to [28].

The vector according to the use of the present invention is preferably characterized by the following features.

[38] The use according to [37], wherein the vector further comprises a temperature sensitive repressor.

[39] The use according to [38], wherein the vector further comprises the temperature sensitive lambda cI857 repressor, a kanamycin resistance gene, origin pBR322, ATP-E ribosomal binding site, and phage fd transcription terminator.

[40] The use according to [37], wherein the temperature inducible promoter is the lambda PR promoter.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

For the avoidance of any doubt it is emphasized that the expressions “in some embodiments”. “in a certain embodiment”, “in certain instances”, “in some instances”, “in a further embodiment”, “in one embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices. In this context, the person skilled in the art will appreciate that the embodiments and features disclosed in the Examples for the recombinant production of an Fc-peptide fusion protein, i.e. romiplostim are intended to be generalized to any Fc-peptide fusion peptide and protein, respectively.

### BRIEF DESCRIPTION OF THE DRAWINGS

FIG. 1: Detailed process flow chart of the established upstream manufacturing process of the Fc-peptide fusion protein (10 L scale bioreactor) according to the present invention.

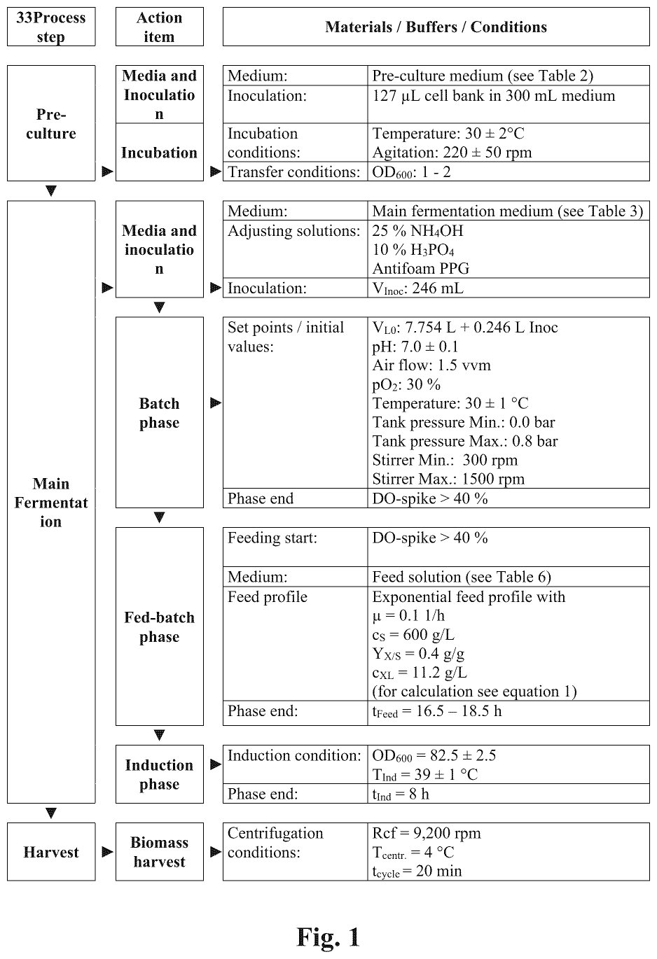

FIG. 2: Comparison of Fc-peptide fusion protein concentration in dependence of the growth rate. Shown is the Fc-peptide fusion protein concentration (c_{P1L}) of fermentation run U56 (μ: 0.1 l/h, OD_{Ind}: 80, T_{Ind}: 39° C., C_{Meth}: 0 g/L) conducted with a growth rate of 0.1 l/h, in comparison to the Fc-peptide fusion protein concentration of fermentation run U60 (μ: 0.2 l/h, OD_{Ind}: 80, T_{Ind}: 39° C., C_{Meth}: 0 g/L) conducted with a theoretical growth rate of 0.2 l/h. The Fc-peptide fusion protein concentration in run U56 increased in the first 8 h of expression time to 10.6 g/L, before decreasing to 8.8 g/L after 10 h of expression time. In the last two hours of the process the product concentration increased again to a final 10.7 g/L. The Fc-peptide fusion protein concentration of fermentation run U60 had already reached its maximum of 10.0 g/L after 4 h of expression time. Afterwards, the concentration decreased to a value of 5.6 g/L at the end of the fermentation. This comparison demonstrates that the growth rate only affects productivity but not maximum Fc-peptide fusion protein concentration.

FIG. 3: Data comparison of the fermentation runs U70 and U69 with and without kanamycin. Black squares and black triangles: fermentation run U69 (OD_{Ind}: 100, T_{Ind}: 39° C.) without kanamycin in the fermentation medium, white squares and white triangles: fermentation run U70 (OD_{Ind}: 93, T_{Ind}: 39° C.) with kanamycin in the fermentation medium, OD₆₀₀: optical density at 600 nm (0-260), c_{PIL}: concentration of the Fc-peptide fusion protein in liquid phase (0-20 g/L). The courses and maximum values of the product concentrations were considered comparable, reaching high concentrations after short time and afterwards decreasing continuously. Therefore, an impact of kanamycin in the fermentation medium on product formation within the specified 6 h of expression time was not observed.

FIG. 4: Offline data of fermentation run U71 (OD_{Ind}: 140, T_{Ind}: 39° C.). OD₆₀₀: optical density at 600 nm (0-310), C_{PIL}: concentration of Fc-peptide fusion protein (Fc-PFP) in liquid phase (0-15 g/L). C_{XL}: cell dry weight (0-80 g/L), C_{P3M}: concentration of acetate in liquid phase (0-30 g/L), C_{SIM}: concentration of glycerol in liquid phase (0-230 g/L). The OD₆₀₀ increased in 21 h of fermentation time to a maximum value of 294. After having reached this maximum, the OD₆₀₀ decreased rapidly in the following 4 h to a temporary minimum of 129 before increasing again in the last two hours to 174 at end of fermentation (EoF). The product concentration reached its maximum after 21 h of fermentation time at 13.5 g/L. Subsequently, the product concentration decreased rapidly to a final value of 4.9 g/L at EoF. Acetate started accumulating in the medium after 19 h of fermentation time, reaching a maximum value of 6.2 g/L after 23 h. Shortly after, glycerol started accumulating in the medium as well. The concentration of glycerol increased constantly to a maximum value of 223.8 g/L at the end of the fermentation.

FIG. 5: Offline data of scale up fermentation run U92 (μ: 0.2 l/h, OD_{Ind}: 80, T_{Ind}: 39° C., C_{Meth}: 0 g/L). OD₆₀₀: optical density at 600 nm (0-260). C_{PIL}: concentration of Fc-peptide fusion protein (Fc-PFP) in liquid phase (0-15 g/L), C_{XL}: cell dry weight (0-100 g/L), C_{P3M}: concentration of acetate in liquid phase (0-20 g/L), C_{SIM}: concentration of glycerol in liquid phase (0-20 g/L). Glycerol concentration decreased in the first 8.7 h from an initial 7.8 g/L. The depletion of glycerol in the fermentation medium marked the end of the batch phase and the start of the feed. Acetate started accumulating in the medium after 17 h of fermentation time. The maximum concentration of 0.8 g/L was reached after 23.2 h of fermentation time, reaching a maximum of 5.8 g/L at EoF. As glycerol was consumed, the OD₆₀₀ started increasing and reached a maximum value of 205 after 17 h of fermentation time. Subsequently, the OD₆₀₀ decreased slightly to a final value of 191 at the end of the fermentation. The cell dry weight (CDW) increased simultaneously with the OD₆₀₀ ending up at a final value of 55.4 g/L. The concentration of Fc-peptide fusion protein increased rapidly after induction and added up to 11.5 g/L after 17 h. In the last two hours of the fermentation, the product concentration remained more or less constant with finally 11.6 g/L.

FIG. 6: Data of fermentation run U75 (u: 0.1 l/h, OD_{Ind}: 80, T_{Ind}: 39° C., C_{Meth}: 0 g/L). pO₂: partial pressure of dissolved oxygen in the medium (0-100%), p: pressure in the fermentation tank (0.0-0.8 bar(g)), Fair: flow rate of inlet air (0-12 L/min), OD₆₀₀: optical density at 600 nm (0-260). C_{PIL}: concentration of Fc-peptide fusion protein in liquid phase (0-15 g/L). After 19.5 h of fermentation time, the stirrer rate had reached its maximum. Subsequently, the pressure in the vessel started to increase automatically from 0.0 to 0.8 bar(g). After 23.8 h the pressure had also reached its maximum value. To prolong the fermentation, the inlet air flow rate was then manually increased from an initial 7.8 to a maximum 11.8 L/min (which corresponds to 1-1.5 vvm, respectively). Thereby it was possible to extend the fermentation by two hours, reaching a total of 9 h of expression time. The OD₆₀₀ increased continuously during the fermentation, reaching a final value of 196 after 26 h of fermentation time. The Fc-peptide fusion protein concentration increased correspondingly, adding up to a maximum value of 7.4 g/L.

FIG. 7: Data of fermentation run U91 (μ: 0.1 l/h, OD_{Ind}: 80, T_{Ind}: 39° C., C_{Meth}: 0 g/L, pressure DO-cascade, constant inlet air flow 1.5 vvm). pO₂: partial pressure of dissolved oxygen in the medium (0-100%), OD₆₀₀: optical density at 600 nm (0-260), C_{PIL}: concentration of Fc-peptide fusion protein (Fc-PFP) in liquid phase (0-15 g/L), C_{XL}: cell dry weight (0-100 g/L), C_{P3M}: concentration of acetate in liquid phase (0-20 g/L), C_{SIM}: concentration of glycerol in liquid phase (0-20 g/L). Throughout the process, OD₆₀₀, CDW and Fc-peptide fusion protein concentration increased continuously. The oxygen limitation during one hour did not have any effect on cell growth and product formation since both kept increasing after the DO had dropped to 0%. The final OD₆₀₀ value added up to 216 and the final Fc-peptide fusion protein concentration reached 6.7 g/L. A potential effect of the oxygen limitation was observed on the CDW. While before DO-limitation CDW added up to 53.0 g/L, it only increased to 54.5 g/L after one hour of limitation. Another effect which was observed was the increase of acetate in the medium. Before the oxygen limitation no acetate concentration could be detected in the medium. After one hour of DO=0% the concentration of acetate had already reached 4.8 g/L.

FIG. 8: Online data of pilot run U93 (μ: 0.1 l/h. OD_{Ind}: 80. T_{Ind}: 39° C. C_{Meth}: 0 g/L, pressure DO-cascade, constant inlet air flow 1.5 vvm). pO₂: partial pressure of dissolved oxygen in the medium (0-100%), ϑ_{L}: temperature in liquid phase (0-50° C.), N_{St}: stirrer agitation (0-1,500 rpm), pH: pH-value of the culture (4-9), Feed: feed rate (0-10 mL/min), p: pressure in the fermentation tank (0.0-0.8 bar(g)), OD₆₀₀: optical density at 600 nm (0-220). The stirrer speed started rising after the DO had reached 30% to keep it at its set point. After 8 h the end of the batch phase was signaled by a sharp rise of DO-signal which automatically triggered the defined exponential feed profile. The induction of the culture was conducted after 18.5 h by increasing/ramping the temperature from 30° C. to 39° C., as the OD₆₀₀ had reached the defined induction range of 80-85. After about 20.5 h the stirrer reached its maximum speed of 1,500 rpm and thus, the pressure in the reactor was increased from 0.0 to 0.75 bar at EoF (maximum possible pressure 0.8 bar) to maintain the DO at 30%. After the previously defined 8 h of expression, the fermentation was terminated, and the fermentation broth was cooled subsequently to 20° C.′.

FIG. 9: Offline data of pilot run U93 (μ: 0.1 l/h, OD_{Ind}: 80, T_{Ind}: 39° C., C_{Meth}: 0 g/L, pressure DO-cascade, constant inlet air flow 1.5 vvm). OD₆₀₀: optical density at 600 nm (0-260). C_{PIL}: concentration of Fc-peptide fusion protein (Fc-PFP) in liquid phase (0-15 g/L), c_{XL}: cell dry weight (0-100 g/L). The OD₆₀₀ of the fermentation increased in the first 18.5 h continuously to a value of 84. After induction, the temperature was maintained at 39° C. during the following 8 h of expression which probably caused the. growth to increase slightly. The final OD₆₀₀ after 26.5 h of fermentation time added up to 202. The cell dry weight (CDW) increased constantly throughout the fermentation and added up to 54.7 g/L at the end of the fermentation. After induction, the Fc-peptide fusion protein concentration increased continuously within 8 h of expression to a final titer of 8.0 g/L.

FIG. 10: Plasmid map of pHIP-Fc peptide fusion protein. The pHIP vector is characterized by the following relevant features: Heat sensitive lambda cI857 repressor, kanamycin resistance gene, origin pBR322, lambda PR promoter, ATP-E ribosomal binding site, phage fd transcription terminator, and Fc-peptide fusion protein gene.

### DETAILED DESCRIPTION OF THE INVENTION

The present invention relates to a process for the recombinant production of an Fc-peptide fusion protein in prokaryotic host cells comprising a temperature induction phase, wherein the temperature induction phase is started through a temperature shift from a first cultivation temperature of about 27° C. to about 35° C. towards a second cultivation temperature of about 38° C. to about 40° C. In particular, the present invention relates to a process for recombinant production of an Fc-peptide fusion protein in prokaryotic host cells, wherein the process comprises a batch phase, a feeding phase before induction, and a temperature induction phase, wherein the prokaryotic host cells, which harbor a temperature inducible expression system as well as a nucleic acid encoding the Fc-peptide fusion protein under control of said temperature inducible expression system, are cultivated during the batch phase and the feeding phase at a first cultivation temperature of about 27° C. to about 35° C. When the cells reach an optical density (OD₆₀₀) of at least 30, the first temperature is increased towards a second temperature of about 38° C. to about 40° C. which results in induction of the expression of the Fc-peptide fusion protein. The second cultivation temperature is maintained for at least about 4 hours.

Accordingly, the process of the present invention is a fed-batch process, which is preferably performed in a bioreactor, wherein feeding is performed before induction as well as during the induction phase. The feeding phase is started when the carbon source is depleted. Thus, a sharp increase of the dissolved oxygen (DO)-signal triggers the start of the feed solution addition, preferably with an exponential feeding profile (see equation 1). Fed-batch protocols result in high cell concentrations, such as in cultures with linearly or exponentially increasing rates of substrate addition before induction (Tabandeh et al., Biotechnology Letters 26 (2004), 245-250)

The term “bioreactor” as used herein refers to any closed vessel used for the growth of prokaryotic microorganisms, but not to laboratory flasks like shake flasks. The bioreactor can be of any size so long as it is useful for the culturing of prokaryotes. Typically, the bioreactor will be at least 1 liter and may be 10, 100, 250, 500, 1000, 2500, 5000, 8000, 10,000, 12,0000 liters or more or any volume in between. The internal conditions of the bioreactor, including, but not limited to pH, DO and temperature, are typically controlled during the culturing period. The bioreactor can be composed of any material that is suitable for holding prokaryotic microorganisms suspended in media under the cultivation conditions of the present invention, including glass, plastic or metal. One of ordinary skill in the art will be aware of and will be able to choose suitable bioreactors for use in practicing the present invention.

Fed-batch processes allow a tight control of the growth phase and can be temporally separated from the production phase, while maintaining plasmid stability and avoiding metabolic stress and production of toxic organic acids. For example, high cell densities and a 23-fold increase in final interferon-gamma concentration in comparison with batch cultures could be achieved in a fed batch process by controlling the substrate feed rate during the growth phase and the postinduction specific growth rate during the production phase (Lim and Jung, Biotechnol. Prog. 14 (1998), 548-553). Curless et al. produced interferon-alpha by initially culturing the cells in a chemostat at 30° C. under glucose limitation. After a steady state was achieved, a fed-batch mode was initiated and the temperature was increased to 42° C. Production of interferon-alpha increased 4-fold under the higher dilution rates tested, further demonstrating the dependence of the pre-induction specific growth rate on productivity (Curless et al., Biotechnol. Prog. 6 (1990), 149-152). In the process of the present invention, the growth rate hardly had an influence on the total Fc-peptide fusion protein yield, and only had an influence on productivity as explained further below.

Product formation can be affected by the chosen feeding strategy (Lee. Trends Biotechnol 14 (1996), 98-105). Feeding strategies such as constant-rate feeding, a stepwise increase of the feeding rate, and exponential feeding have been used to obtain high cell-densities of E. coli in fed-batch cultures. Exponential feeding is a simple but efficient method that has been successfully used for the high cell density cultivation of several non-recombinant and recombinant E. coli strains. In the process of the present invention, an exponential feeding strategy is preferably performed, preferably with the feeding profile as defined in equation 1.

As can be derived from the Examples, the production process was established for the Fc-peptide fusion protein romiplostim, which is a thrombopoiesis-stimulating peptibody being composed of an Fc domain of IgG1 fused to a peptide domain that mimics the function of endogenous thrombopoietin (TPO). Fc-peptide fusion proteins comprise a common structure, i.e. in frame fusion of an active high-affinity peptide to an Fc region. In particular, the term “Fc-peptide fusion” means the gene fusion of a DNA encoding a target peptide to the DNA encoding the Fc-moiety, i.e. CH2/CH3 domains, of an immunoglobulin, expressing one single polypeptide. The term “Fc” refers to the Fc domain, the crystallisable fragment of an antibody.

In the 1980s, nearly all peptides entering clinical development were less than 10 amino acids long. Average peptide length has increased in each subsequent decade, largely due to improvements in peptide synthesis and manufacturing technology. Currently, development candidates are more equally distributed in the various length ranges up to 40 amino acids, suggesting that length is no longer a serious limitation for peptide drug development (Lau and Dunn. Bioorganic & Medicinal Chemistry 26 (2018), 2700-2707). Accordingly, the term “peptide” refers to an amino acid sequence having less than 100 amino acids, preferably less than 50 amino acids, preferably between 10 and 40 amino acids, most preferably between 10 and 30 amino acids, for example 14 amino acids. The terms “Fc-peptide fusion protein”. “Fc-fusion peptide”, “Peptide-Fc fusion protein” and “peptibody” are used interchangeably herein and refer to recombinant polypeptides engineered by fusing a gene coding for a biologically active peptide in frame with the gene coding for an Fc domain resulting in a peptide-Fc chimera with antibody-like structure. The term “Fc-peptide fusion protein” does not include full length proteins fused to an Fc domain.

Since Fc-peptide fusion proteins comprise a common structure, i.e. in frame fusion of an active high-affinity peptide to the Fc region of an immunoglobulin, it is prudent to expect that the production process of the present invention, which has been established for romiplostim, is applicable for Fc-peptide fusion proteins in general and in particular to those which are produced in prokaryotic host cells. Accordingly, when reference is made to an Fc-peptide fusion protein, in particular by way of reference to the Examples, it is not intended to limit the scope of the present invention to the Fc-peptide fusion protein romiplostim.

An Fc-peptide fusion protein is a biopharmaceutical and biopharmaceuticals are the main drugs developed in the pharma sector. Advancements in the area of recombinant protein production have changed the previous trend, making the yield much higher and the cost much lower, thus allowing the production of such proteins on an industrial scale and opening the door for the treatment of multiple diseases and disorders. With the help of recombinant protein technology, expression of recombinant protein-based biopharmaceuticals has been achieved using prokaryotic organisms. Exemplarily prokaryotic hosts that are used in accordance with the present invention include but are not limited to species of Escherichia, Bacillus, Pseudomonas, Streptomyces, and Corynebacterium, in particular to Escherichia coli. Bacillus subtilis, Pseudomonas fluorescens, Streptomyces spp, and Corynebacterium glutamicum.

E. coli is one of the organisms of choice for the production of recombinant proteins. Its use as a cell factory is well-established and it has become the most popular expression platform. Accordingly, in a preferred embodiment, the prokaryotic host used in accordance with the present invention is E. coli. The most important E. coli strains used for the production of biopharmaceuticals are E. coli BL21 and E. coli K12 as well as derivatives of said lineages. However, there are dozens of different E. coli strains that can be used in accordance of the present invention; see for example www.openwetware.org/wiki/E._coli_genotypes, which lists various E. coli strains which are all incorporated herein by reference. In a preferred embodiment, E. coli BL21 as well as derivatives thereof are used in accordance with the present invention. In a particular preferred embodiment. E. coli BL21 (F⁻ ompT gal dcm lon hsdS_{B}(r_{B}⁻ m_{B}⁻) [malB⁺]_{K-12}(λ^{S})) is used in accordance with the present invention. This strain is galactose non-utilizing, deficient in Lon and OmpT proteases, and the “malB region” was transduced in from the K-12 strain W3110 to make the strain Mal⁺λ^{S}; see for example Studier et al. (2009) J. Mol. Biol. 394(4), 653.

Prokaryotic microorganisms grow by using a variety of carbon sources, for example carbon dioxide (photoautotroph and chemoautotroph), or organic compounds (photoheterotroph and chemoheterotroph). Accordingly, in one embodiment, the culture medium as used in accordance with the present invention either comprises an organic carbon source or an inorganic carbon source. In a preferred embodiment, the prokaryotic microorganisms are heterotroph, and an organic carbon source is used for cultivation. There are at least about 200 carbon sources known which support the growth of prokaryotic microorganisms and which can be used in accordance with the present invention, some of which include but are not limited to L-arabinose, N-acetyl-D-glucosamine, D-saccharic acid, succinic acid, D-galactose, L-aspartic acid, L-proline, D-alanine, D-trehalose, D-mannose, dulcitol, D-serine, D-sorbitol, glycerol, L-fucose, D-glucuronic acid, D-gluconic acid, D,L-α-glycerol-phosphate, D-xylose, L-lactic acid, formic acid, D-mannitol, L-glutamic acid, glucose 6-phosphate, D-galactonic acid γ-lactone, D,L-malic acid, D-ribose, tween 20, L-rhamnose, D-fructose, acetic acid, α-D-glucose, maltose, D-melibiose, thymidine, L-asparagine, D-aspartic acid, D-glucosaminic acid, 1,2-propanediol, tween 40, α-keto-glutaric acid, α-keto-butyric, α-methyl-D-galactoside, α-D-lactose, lactulose, sucrose, uridine, L-glutamine, m-tartaric acid, glucose 1-phosphate, fructose 6-phosphate, tween 80, α-hydroxy-glutaric acid γ-lactone, α-hydroxy-butyric acid, β-methyl-D-glucoside, adonitol, maltotriose, 2′-deoxyadenosine, adenosine, glycyl-L-aspartic acid, citric acid, m-inositol, D-threonine, fumaric acid, bromo-succinic acid, propionic acid, mucic acid, glycolic acid, glyoxylic acid, cellobiose, inosine, glycyl-L-glutamic acid, tricarballylic acid, L-serine, L-threonine, L-alanine, L-alanyl-glycine, acetoacetic acid, N-acetyl-β-D-mannosamine, mono-methyl succinate, methyl pyruvate, D-malic acid, L-malic acid, glycyl-L-proline, p-hydroxy phenylacetic acid, m-hydroxy phenylacetic acid, tyramine, D-psicose, L-lyxose, glucuronamide, pyruvic acid, L-galactonic acid γ-lactone, D-galacturonic acid, phenylethylamine, and 2-amino ethanol.

Glucose is the most common carbon source for the cultivation of prokaryotic microorganisms, and in particular for cultivation of E. coli. Accordingly, the culture medium can comprise glucose as carbon source. As a by-product of biodiesel production, glycerol is expected to become a sustainable alternative substrate to commonly used glucose. Thus, in a preferred embodiment, the culture medium used in accordance with the present invention comprises glycerol as carbon source. The concentration of the carbon source can vary between about 1 g/L to about 10 g/L and in a preferred embodiment, the culture medium as used in accordance with the present invention comprises about 5 g/L of the carbon source, in particular 5 g/L glycerol.

In general, culture media for production processes using prokaryotic microorganism are known in the art. For example, the utilization of complex-rich media with glycerol or glucose as carbon source, that are supplemented with yeast extract, peptone or tryptone, alone or mixed (Tabandeh et al. 2004, supra; Harrison et al., Biotechnol Bioeng 53 (1997), 611-22), and protease inhibitors (Harrison et al. 1997, supra; Yoon and Kang, Biotechnol Bioeng 43 (1994), 995-999) are commonly used in fed-batch processes for the production of recombinant proteins in E. coli and thus, can also be used in accordance with the present invention. In a preferred embodiment, the culture medium as used in accordance with the present invention comprises glycerol, preferably 5 g/L; yeast extract, preferably 20 g/L; L-methionine, preferably 2 g/L; NaCl, preferably 1 g/L; KH₂PO₄, preferably 6.9 g/L; K₂HPO₄, preferably 8.7 g/L: citric acid, preferably 3.5 g/L; MgSO₄×7 H₂O, preferably 2 g/L; Thiamine HCl, preferably 5×10⁻³ g/L; Antifoaming agent, preferably Antifoam PPG, preferably 0.2 g/L; and a trace element solution, preferably 10 ml/L which includes MnCl×2 H₂O, preferably 1.64 g/L; ZnCl₂, preferably 1.48 g/L; CoCl₂×6 H₂O, preferably 0.27 g/L; FeSO₄×7 H₂O, preferably 3.25 g/L: CuSO₄×5 H₂O, preferably 0.22 g/L; H₃BO₃, preferably 0.34 g/L; Na₂MoO×2 H₂O, preferably 0.27 g/L; Ethylenedinitrilotetraacetic acid disodium salt dehydrate, preferably Titriplex® III, preferably 0.63 g/L; and HCl, preferably 20 mL/L of 10% HCl.

The pH is regulated by acid/base addition and is kept constant during the different cultivation phases. In particular, the pH is between pH 6.5 and 7.5, preferably 7.0±0.5, most preferably 7.0±0.1.

As mentioned above, among the underlying reasons for a widespread use of E. coli in the production of biopharmaceuticals is the availability of a variety of strong inducible promoters. The promoters commonly used for heterologous protein expression require the addition of an inducer molecule, the depletion or addition of a nutrient, or a shift in a physical or physicochemical factor, such as pH. However, each of these options bears different disadvantages which may complicate the bioprocess and increase its cost (Valdez-Cruz et al. 2010, supra). Chemical inducers, such as IPTG and antibiotics, can be expensive and toxic, and have to be eliminated in the final product or in the waste effluents of the bioprocess, which requires additional controls and downstream operations. In expression systems based on nutrient exhaustion, the control of the induction timing is difficult, and starvation can affect cell metabolism or synthesis of the recombinant protein. In the case of pH-inducible expression systems, there are only few vectors available, and the pH for induction can depart from the optimal pH for physiological conditions (Valdez-Cruz et al. 2010, supra).

Many of these disadvantages can be overcome by the use of thermoregulated expression systems (Makrides. Microbiological Reviews 60 (1996), 512-538) as used in accordance with the present invention. A temperature inducible expression system that has been widely used to produce recombinant proteins and peptides in prokaryotic cells is based on the strong major leftward (pL) and/or rightward (pR) phage lambda promoters which are finely regulated by the mutant thermolabile cI857 repressor of bacteriophage lambda (Villaverde et al. 1993, supra). Gene expression is inhibited at cultivation temperatures below 37° C. (normally in the range of 28-32° C.) whereas transcription by the host RNA polymerase ensues upon inactivation of the mutant thermolabile cI857 repressor by increasing the cultivation temperature, usually to 42° C. By using the temperature inducible expression system, the use of special cell culture media, toxic or expensive chemical inducers is avoided, and culture handling and contamination risks are minimized. Furthermore, the temperature inducible expression system is easily scalable as temperature in bioreactors can be readily modified by external means (Valdez-Cruz et al. 2010, supra).

Accordingly, the temperature inducible promoter as used in accordance with the present invention is the lambda PR promoter, which is regulated by a temperature sensitive repressor, preferably the lambda cI857 repressor.

Various cultivation parameters, such as cell culture media composition, pH, agitation, aeration, temperature, cell density, induction time, and feeding strategies affect the protein expression level depending upon expression systems. Thus, it is important to evaluate the cultivation conditions for the expression of at least every class of recombinant proteins, if not of every type of recombinant protein expressed as they can affect recombinant protein productivity, and to evaluate the development of effective bioprocesses.

As mentioned above, cultivation of the prokaryotic microorganism (the first cultivation temperature) in accordance with the present invention is performed at about 27° C. to about 35° C., but not higher than 37° C. In a preferred embodiment, the first cultivation temperature is 30° C.±1° C., i.e. 29° C. to 31° C., preferably 30° C.

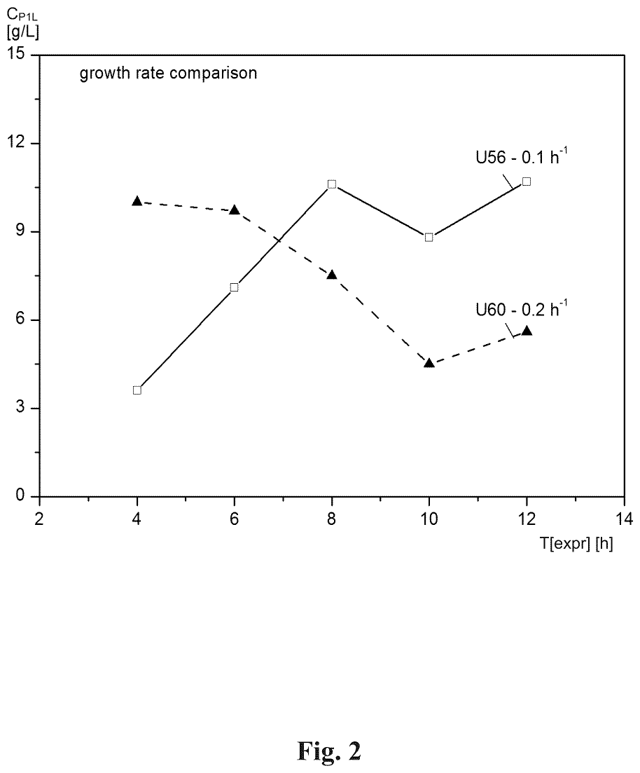

In a first phase a process strategy was developed at 1 L scale to identify the process parameter settings with the highest product yield within a certain parameter range. Of the four tested parameters OD_{Ind}, μ, T_{Ind} and Cₘₑₜₕ, the induction OD₆₀₀ and the induction temperature showed the most significant influence on Fc-peptide fusion protein yield. The growth rate hardly had an influence on the total Fc-peptide fusion protein yield, and only had an influence on productivity, given that comparable maximum Fc-peptide fusion protein titers were reached with both 0.1 and 0.2 l/h, but in an 8 h shorter process time with a growth rate of 0.2 l/h: see Example 1 and FIG. 2.

Furthermore, scale up experiments in a 10 L bioreactor showed that no statistical connection between the factor variance of OD_{Ind}, F_{Feed} and pH to Fc-peptide fusion protein yield was detected and that the process of the present invention is robust regarding process parameter variations of these three factors tested in the investigated area. However, a statistical connection between Fc-peptide fusion protein yield and induction temperature deviation was found, leading to the conclusion that this specific process parameter has to be closely controlled and monitored to ensure optimal Fc-peptide fusion protein production.

Heating strategies, such as time and temperature of induction, are among the most important factors that have to be considered and controlled to improve the productivity of heat-inducible expression systems. A variety of heating strategies have been developed to avoid the adverse effects of high temperature, such as decreased growth rate, damage to the host cells, decrease in viability and productivity, and plasmid instability. Commonly, the expression is induced through an increase in cultivation temperature from 28-32° C. to about 42° C. In some cases, a few minutes after the temperature has been raised, it is decreased back to 38° C. to 40° C. For example. Tabandeh et al. induced cultures at 42° C. for 20 or 40 min and then decreased the temperature to 37° C. for 4 h. The authors reported that the recombinant protein was degraded when the induction phase at 42° C. lasted 40 min, whereas degradation was absent if temperature was decreased to 37° C. within 20 minutes after induction (Tabandeh et al. 2004, supra).

As can be derived from Example 1, surprisingly, the fermentations with a lower induction Temperature of 39° C. resulted in a higher Fc-peptide fusion protein concentration than those with an induction temperature of 42° C., i.e. the temperature which is commonly used for temperature inducible protein expression. Accordingly, as already mentioned above, the first temperature is increased towards a second temperature of about 38° C. to about 40° C. in the process of the present invention, which results in induction of the expression of the Fc-peptide fusion protein. In a preferred embodiment, the first temperature is increased towards a second temperature of about 39° C. In a particular preferred embodiment, the first cultivation temperature is 30° C. and the second cultivation temperature is 39° C. in the process of the present invention. Furthermore, it has been surprisingly observed that the maintenance of the second temperature for longer than 20 or 40 minutes, i.e. for at least 4 hours results in a high Fc-peptide fusion protein concentration. In a preferred embodiment, the second temperature is maintained at least for 5 to 10 hours, preferably for 6 to 8 hours, more preferably for about 6 or about 8 hours, and most preferably for about 8 hours.

For the temperature change, a uniform distribution is achieved by a so-called disk blade stirrer, on which three disk blade elements are arranged at the bottom, in the middle and at the top of the fermenter. Accordingly, in one embodiment, a uniform temperature distribution is achieved by a disk blade stirrer. For the measurement of the temperature a standard PT100 element is used, which has a measuring point in the medium.

A further factor that has an impact on productivity of heat-inducible expression systems is the beating rate. Caspeta et al. investigated heating rate differences in relation to scale. Heating rates of 6, 1.7, 0.8, and 0.4° C./min, typical of 0.1, 5, 20, and 100 m³ bioreactors, respectively, were simulated in a laboratory scale bioreactor. The authors concluded that the maximum recombinant protein production and minimum accumulation of waste organic acid by-products was obtained during the slowest heating rates that emulated the largest scale bioreactors. The results demonstrated that during faster heating rates, typical of laboratory conditions, the cells required more energy and experienced larger imbalances between glycolysis and the TCA cycle than during slower heating rates characteristic of large-scale vessels. The study also demonstrated that cells subjected to slow heating rates can better adapt to thermal stresses than those exposed to a faster temperature increase (Caspeta et al., Biotechnol Bioeng 102 (2009), 468-82). In the experiments performed in accordance with the present invention usually a heating rate of 3.6° C./min has been employed in 10 L bioreactors. Accordingly, in one embodiment, the heating rate to shift the first cultivation temperature towards the second cultivation temperature in the process of the present invention is between 1° C./min and 6° C./min, preferably between 1° C./min and 4° C./min, more preferably 3.6° C./min for a 10 L bioreactor. Of course, the person skilled in the art knows that the corresponding heating rates should be adapted when using larger scale bioreactors as for example described in Caspeta et al. 2009, supra.

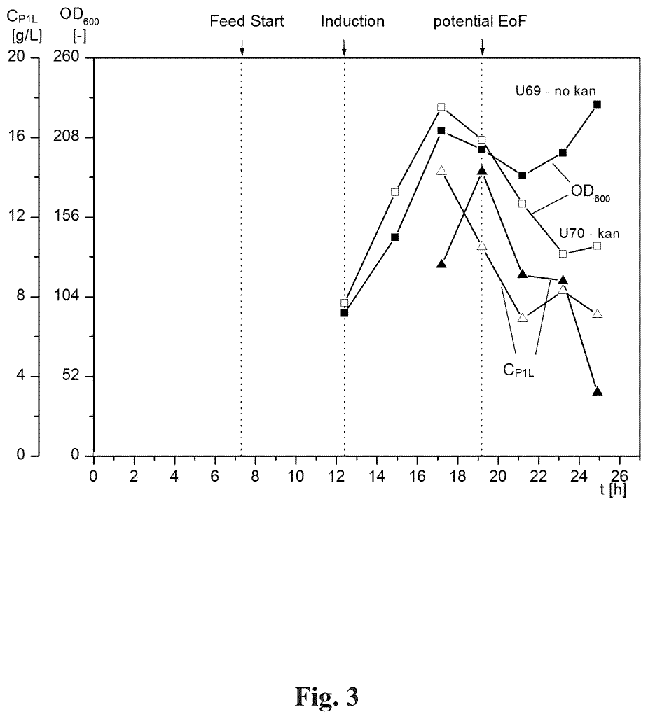

According to the experiments performed within the scope of the present invention, induction, i.e. start of the temperature shift, at an OD₆₀₀ of 30 resulted in a reasonable concentration of the Fc-peptide fusion protein as can be derived from Example 1. The fermentation runs with an induction OD₆₀₀ of 80 (U60) resulted in a significantly higher maximum product concentration than the runs with an induction OD₆₀₀ of 30. Fermentation runs U69 to U71 were induced at OD₆₀₀>80, in particular at OD₆₀₀ of 95±5 (U69 and U70) and at OD₆₀₀ of 140 (U71) and achieved 35 to 40% higher maximum Fc-peptide fusion protein yields than fermentation run U60 which was induced at an OD₆₀₀ of 80; see Example 2 and FIG. 3. Accordingly, the induction in the process of the present invention is started between OD₆₀₀ of 30 and 140. However, given that the maximum product concentration of run U71 was 6% lower than in the runs U69 and U70, the induction in the process of the present invention is preferably started between OD₆₀₀ of 30 and 100. However, in order to ensure a robust process design regarding its time point of induction, it is most preferred to perform the induction at an OD₆₀₀ of 80±5, preferably of 82.5±2.5.

In one embodiment, the cell culture medium used in the batch phase and the feeding phase can be the same, i.e. as defined above. In another embodiment, the feeding medium only comprises glycerol, preferably 300 g/L; L-methionine, preferably 2.5 g/L; yeast extract, preferably 150 g/L; K₂HPO₄, preferably 5.3 g/L; and KH₂PO₄, preferably 2 g/L.

The methionine concentration in the feed solution did not show any effect on product formation as can be derived from Example 1. Accordingly, a cell culture medium with less ingredients than usual can be used in the process of the present invention and thus, omitting methionine in the cell culture medium during the feeding phase results in cost reduction. Accordingly, in one embodiment, the feeding medium (either the one also used in the batch phase or the feeding medium defined herein before) does not comprise L-methionine.

As will be explained further below, the vector harboring the gene encoding the Fc-peptide fusion protein further comprises a selectable marker, which is in the present case an antibiotic resistance gene, in particular the gene conferring kanamycin resistance. To avoid the loss of the plasmid during cultivation, the cultivation medium is usually supplemented with the respective antibiotic, here for example kanamycin. However, as can be derived from the experiments performed in accordance with the present invention, the addition of kanamycin as tested in the fermentations U69 and U70 did not show any influence on Fc-peptide fusion protein yield or cell growth within the relevant process time. This is advantageous since the use of antibiotics in a biopharmaceutical production process can be expensive and the antibiotics have to be eliminated in the final product and/or in the waste effluents of the bioprocess, which requires additional controls and downstream operations.

Accordingly, in one embodiment, the medium used in the batch phase of the process of the present invention does not comprise any antibiotic, and preferably not kanamycin. In another embodiment, the medium used in the feeding phase of the process of the present invention does not comprise the antibiotic. In another embodiment, the medium used in the batch phase and the feeding phase of the process of the present invention does not comprise the antibiotic.

Furthermore, a negative impact on cell growth and product formation was observed while reducing the dissolved oxygen (DO) set point from 30% to 8% (see Example 3. Table 18). However, one hour of DO-limitation after 8 h of expression did not show any negative effect on cell growth and product formation. Accordingly, in one embodiment, the DO concentration is maintained at above 8%, preferably between 8% and 30% in the process of the present invention. More preferably, the DO concentration is maintained at above 10%, preferably between 10% and 30% and most preferably, the DO concentration is 30% in the process of the present invention. “Maintaining” means in this context, that the DO concentration does not drop under the mentioned values for longer than about two hours, preferably no longer than about one hour.

Means and methods for controlling and maintaining a specific DO concentration in a bioreactor. i.e. means and methods for aeration, are known to a person skilled in the art. For example, the oxygen supply can be varied by manipulating air flow rate, oxygen content in the incoming air, reactor pressure, and stirrer speed and the DO concentration is measured with commercially available DO sensors. In one embodiment, the DO concentration in the process of the present invention is maintained by adjusting the stirrer speed, preferably increasing the stirrer speed from 300 rpm to 1,500 rpm in the course of cultivation, by aeration, preferably with a constant air flow of 1 vvm to 2 vvm, preferably of 1 vvm or 1.5 vvm or by an increasing air flow from 1 vvm to 2 vvm, preferably from 1 vvm to 1.5 vvm, and by increasing the tank pressure in the course of cultivation, preferably from 0.0 to 0.8 bar(g).

Preferably, the pressure in the bioreactor is increased, preferably up to 0.8 bar to maintain the DO at 30%, i.e. to ensure sufficient oxygen supply, after the stirrer rate and/or the air flow had reached its maximum.

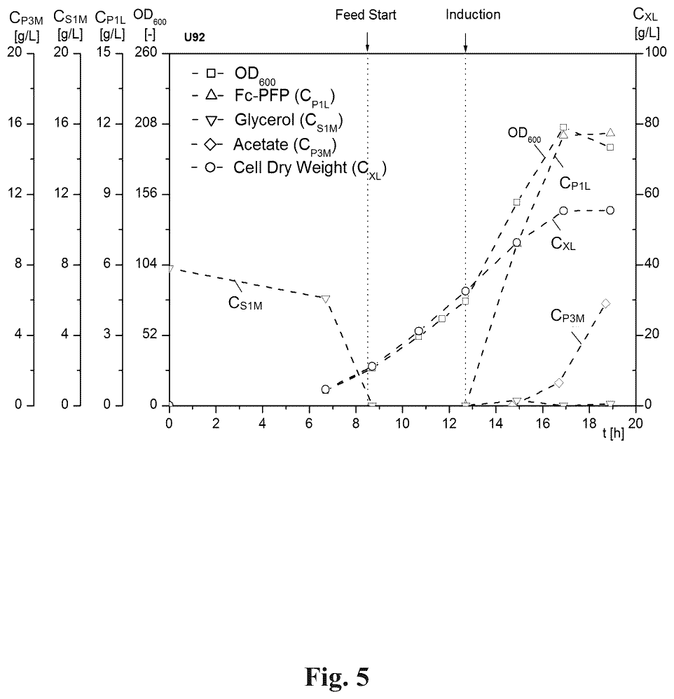

After identifying optimal cultivation conditions, i.e. conditions for an efficient Fc-peptide fusion protein production, the developed process strategy was scaled up to 10 L scale in a second development phase. The scale up runs were considered successful, given that all fermentations showed comparable or higher maximum Fc-peptide fusion protein concentrations and similar cell growth courses compared to the reference run in 1 L scale. Furthermore, the reproduction of the process strategy showed no technical issues in 10 L scale; see Example 4 and FIG. 5. Thus, the data obtained within the course of the experiments performed in accordance with the present invention are based on bioreactor experiments, which allows further extrapolation to large scale fermentation processes. Accordingly, the process of the present invention can be used for Fc-peptide fusion protein production at large scale. In one preferred embodiment, the process of the present invention is a large scale process.

“Large scale”, also called “production scale” or “manufacturing scale” or “commercial scale”, when used according to the process of the invention refers to Fc-peptide fusion protein production in prokaryotic cells at a scale higher than 100 L, for example 200 L scale and higher.

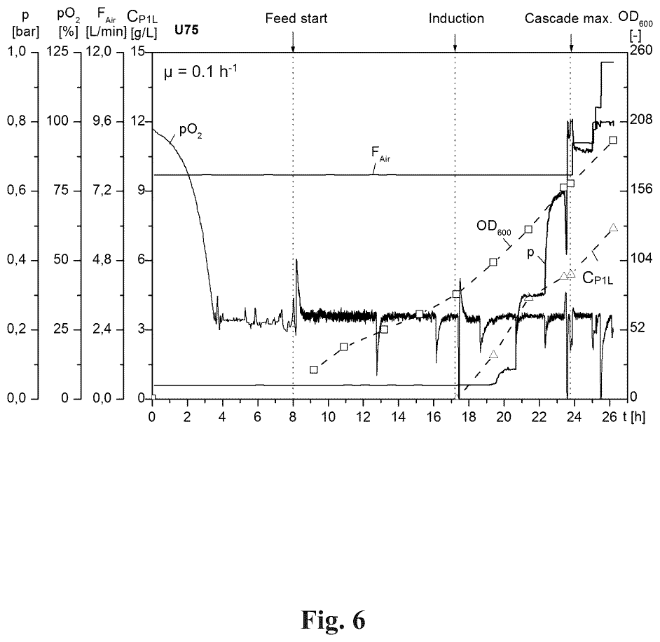

When cultivating E coli, the growth rate is usually between 0.1 and lower, and up to about 0.3 l/h to avoid acetate formation (Lee (1996), supra). Accordingly, in one embodiment, the growth rate of the prokaryotic host cell in the process of the present invention is between 0.05 and 0.3 l/h. As can be derived from Example 1 and as also explained hereinbefore, a growth rate of 0.2 l/h resulted in a high productivity. Accordingly, in one embodiment, the growth rate of the prokaryotic host cell in the process of the present invention is 0.2 l/h. In further experiments performed within the scope of the present invention, the growth rate was changed to 0.1 l/h to ensure slower oxygen consumption by the cells. These adaptations were tested in fermentation run U75 (see Example 5, FIG. 6) which was considered technically feasible and therefore successful although 25% lower Fc-peptide fusion protein concentration within an 11 h extended process time were achieved compared to the previous process strategy. Accordingly, in one embodiment, the growth rate of the prokaryotic host cell in the process of the present invention is 0.1 l/h. In summary, the growth rate of the prokaryotic host cell in the process of the present invention can vary and is preferably in the range between 0.05 and 0.2 l/h.

In further experiments performed within the scope of the present invention, influences of a filter sterilized feed solution during expression were investigated. The sterilization of the feed solution via 0.2 μm filter in run U73 showed comparable results to the fermentation with a heat sterilized feed solution (U72). Accordingly, the feed solution as used in the process of the present invention is either heat sterilized or filter sterilized, but preferably filter sterilized.

In one embodiment, the process of the present invention comprises the harvest of the cells from the culture medium. Methods for cell harvesting are known to a person skilled in the art and can include but are not limited to centrifugation and filtration, or a combination of both. An overview of common harvesting technologies is given for example in Turner et al., Adv Biochem Eng Biotechnol 165 (2018), 95-114. In a preferred embodiment of the present invention, the cells are harvested by centrifugation, more preferably by centrifugation for 20 min at 9,200 g and 4° C.

In one embodiment, the process of the present invention further comprises a downstream step which includes the harvest of the Fc-peptide fusion protein from the prokaryotic host cells. Overexpression of heterologous recombinant polypeptides in transformed microorganisms often results in the formation of so-called inclusion bodies (IBs), which contain the recombinant protein in non-native form. These inclusion bodies are highly refractile, amorphous aggregates and the polypeptides therein are generally unfolded, reduced, inactive, and at least partially insoluble in common aqueous buffers. Same applies to the Fc-peptide fusion protein as produced with the method of the present invention. Accordingly, in one embodiment, the Fc-peptide fusion protein as produced with the process of the present invention is expressed in insoluble or limited soluble form and is accumulated in inclusion bodies in the cytoplasma of the host cell. In general, the increased cultivation temperature (second temperature) and high production rates alter protein folding, which in turn favors protein aggregation into inclusion bodies (IB) when using a temperature-inducible expression system, for example the lambda pL/pR-cI857 system (Valdez-Cruz et al. (2010), supra). Accumulation of the recombinant protein in non-native form in IB has certain advantages, as rapid intracellular degradation is avoided (Caspeta et al. (2009), supra) and it can be isolated and concentrated by a simple centrifugation step, reducing the downstream processing costs and facilitating the production of toxic proteins to cells (Valdez-Cruz et al. (2010), supra) However, a refolding step is then required to recover a biologically active protein.

Processes for obtaining recombinant proteins from inclusion bodies are described in the art and generally comprise lysis and disruption of the cells followed by centrifugation. The pellet comprising a large proportion of inclusion bodies is usually washed with detergents to remove lipid membranes, lipopolysaccharides (LPS), other cell debris, and other contaminants. The scientific literature provides many methods to isolate and purify inclusion bodies and to solubilize and refold the recombinant protein afterwards into its native state; see for example Cabrita and Bottomley, Biotechnology Annual Review 10 (2004), 31-50 and Singh and Herzer, Adv Biochem Eng Biotechnol 165 (2018), 115-178. Furthermore, various purification protocols are available; see for example GE Healthcare. Application note 18-1112-33 AC, Protein purification. An overview is also provided in international application PCT/EP2021/086384 at page 2, line 27 to page 6, line 7, which content is herein incorporated by reference. As regards the Fc-peptide fusion protein romiplostim, Linderholm and Chamow, BioProcess International 12 (2014), 30-35 refer to a typical purification scheme which includes centrifugation/filtration to yield a clarified supernatant followed by protein A chromatography before polishing with additional chromatography steps. Zhang et al., Eng Life Sci 20 (2020), 422-436 describe a purification procedure of cation exchange and hydrophobic chromatography to first reduce endogenous host cell proteins prior to Protein A chromatography. WO 2000/024770 A2 and WO 2000/024782 A2 describe the purification of an Fc-TPO mimetic peptide (Fc-TMP-TMP) and Fayaz et al., Daru. 14:18 (2016). doi: 10.1186/s40199-016-0156-7 describe methods for cell lysis, separation, denaturation, solubilization, refolding and purification by Protein A Sepharose affinity chromatography in section “Purification of romiplostim recombinant protein”. WO 2017/168296 A1 discloses the purification of an Fc-fusion protein by using a series of steps comprising Protein A as capture step, followed by anion exchange chromatography and hydrophobic interaction chromatography, prior to cation exchange chromatography as final polishing step. All of the above-mentioned methods are herein incorporated by reference.

Accordingly, in one embodiment, the process of the present invention further comprises a step of isolation of the Fc-peptide fusion protein containing inclusion bodies, preferably by lysis/disruption of the cells and a sedimentation step, preferably a centrifugation step. In one embodiment, the process of the present invention further comprises solubilization of the inclusion bodies. In one embodiment, the process of the present invention further comprises a step of refolding the Fc-peptide fusion protein. In one embodiment, the process of the present invention comprises a further downstream step, i.e. a step of purifying the Fc-peptide fusion protein. This can be performed by methods known to a person skilled in the art, for example including but not limited to chromatographic methods, filtration, buffer exchange, etc., see above.

In a preferred embodiment, the step of purifying the Fc-peptide fusion protein in the process of the present invention (for example to remove a sulfide variant of the Fc-peptide fusion protein, which for example comprises at least one mismatched disulfide bond) is performed as follows, wherein the steps are performed in the indicated order:
i) performing an affinity capture chromatography in bind-elute mode:
ii) performing a mixed-mode chromatography in bind-elute mode:
iii) performing a cation exchange chromatography in bind-elute mode; and
iv) performing an ultrafiltration/diafiltration:
wherein the chromatographic steps a), b), and c) are performed optionally with one or more washing steps.

In one embodiment, the process of the present invention comprises elution of the Fc-peptide fusion protein from the affinity chromatography medium of step i) with a decreasing linear pH gradient, which preferably starts at about pH 5.5 and ends at about pH 2.5. In one embodiment, step i) of the process of the present invention is performed with a Protein A chromatography medium, which preferably comprises an alkali-tolerant Protein A derivative as a ligand, preferably an alkali-stabilized tetramer variant of domain B of Protein A bound to a cross-linked agarose matrix. In one embodiment, at least one of the wash and the elution buffer used in the Protein A chromatography of step i) of the process of the present invention includes urea, preferably both the wash and the elution buffer include urea. In one embodiment, in step i) of the process of the present invention, the eluate of the Protein A capture chromatography is collected in stabilizing buffer, wherein the stabilizing buffer preferably comprises D-mannitol, sucrose, L-histidine, and Tween 20. In one embodiment, the pH of said stabilizing buffer is in the range of 3.5 to 6.5, preferably 4 to 6, more preferably between 4.5 and 5.5, most preferably about 5. In one embodiment, step ii) of the process of the present invention is performed with a positively charged mixed-mode chromatography medium, wherein the Fc-peptide fusion protein is preferably eluted from said positively charged mixed-mode chromatography medium with a decreasing pH gradient, which is preferably formed by mixing of two buffers having pH values of about pH 5 and about pH 7. In one embodiment, the positively charged mixed-mode chromatography medium used in accordance with the process of the present invention comprises N-benzyl-N-methyl ethanolamine as a ligand bound to a highly cross-linked agarose matrix. In one embodiment, the mixed-mode chromatography of step ii) of the process of the present invention is preceded by a conditioning step, preferably wherein the conditioning step comprises dilution, pH adjustment, incubation, and filtration of the eluate of the affinity capture chromatography of step i). In one embodiment, step iii) of the process of the present invention is performed with a strong cation exchange medium, preferably with charged groups -R-SO3-bound to a hydrophilic polymer matrix. In one embodiment, the Fc-peptide fusion protein is eluted from the strong cation exchange medium by an increase of the pH value in the process of the present invention. The details and corresponding Examples supporting the above-mentioned embodiments are described in international application PCT/EP2021/086384 and are herein incorporated by reference. In particular, it is understood that all embodiments of any one of claims 1 to 26 of international application PCT/EP2021/086384 can be combined with the embodiments of any one of items [1] to [40] and appended claims 1 to 23, respectively.

As mentioned above, Fc-peptide fusion proteins combine the biologic activity of peptides with the stability of monoclonal antibodies, wherein the active high-affinity peptide is fused in frame to the Fc region of an immunoglobulin. The five primary classes of immunoglobulins are IgG, IgM, IgA, IgD and IgE, wherein IgG is most abundant in human serum. Four subclasses of IgGs exist, i.e. IgG1, IgG2, IgG3, and IgG4. In a preferred embodiment, the fused Fc domain is derived from human immunoglobulin, preferably from IgG, more preferably from IgG1. The peptide sequences are usually isolated from phage libraries or known sequences and integrated by recombinant cloning techniques. The peptide represents the biologically active region and is typically affinity matured and optimized for the desired biological activity. Accordingly, in one embodiment of the present invention, the peptide comprises at least one binding sequence. Improvement of the affinity of the peptide can be achieved by duplication or alteration of the peptide sequence or addition of specifically designed flanking spacers, residues, or linker sequences. The orientation of the peptide sequence can alter its activity, e.g., certain peptides seem to be more active when fused to the carboxy terminus of the Fc (Shimamoto et al. (2012), supra). Thus, in one embodiment, the peptide of the Fc-peptide fusion protein as produced by the process of the invention is fused at the C-terminus of a human immunoglobulin-Fc, preferably of human IgG1-Fc. The length of the peptide fused to the monomeric Fc chain can range from 15 to 100 amino acids, preferably from 25 to 70 amino acids, most preferably from 30 to 50 amino acids.

Furthermore, the homodimerization of two Fc moieties provides a minimum of two peptides per peptibody, thereby increasing the avidity for its target. Fc-peptide fusion proteins can also be modified to polymerize into well-defined complexes containing for example twelve fused partners. Different capabilities for different Fc-fusion stoichiometries are for example described in Czajkowsky et al., EMBO Mol Med. 4 (2012), 1015-1028. Accordingly, in one embodiment, the Fc-peptide fusion protein as produced by the process of the invention is a monomer, dimer, or polymer, preferably a dimer.

One exemplarily peptibody is romiplostim (DrugBank Accession Number DB05332, Nplate®, AMG531, WO 2000/024770 A2, WO 2000/024782 A2) which was approved for the treatment of immune thrombocytopeniarpura (ITP) by the United States Food and Drug Administration (FDA) in 2008 and by the European Medicines Agency (EMA) in 2009, respectively. ITP is an autoimmune disorder characterized by platelet deficiency due to platelet destruction and/or insufficient production (Fayaz et al. (2016), supra). The thrombopoiesis-stimulating peptibody romiplostim is composed of an Fc domain of IgG1 fused to a peptide domain that mimics the function of endogenous thrombopoietin (TPO). Through binding to the thrombopoietin receptor TPOR (also known as c-MPL) on platelets and platelet precursors romiplostim acts as a TPO receptor agonist and activates intracellular transcriptional pathways to increase the production of platelets. Thus, in one embodiment the Fc-peptide fusion protein as produced by the process of the present invention is a receptor agonist, preferably wherein the receptor agonist is a thrombopoietin mimetic. In other words, in one embodiment, the peptide of the Fc-peptide fusion protein as produced by the process of the invention comprises an amino acid sequence that is a receptor agonist, preferably a mimetic of a natural occurring receptor agonist, more preferably, a thrombopoietin mimetic, i.e. the peptide has a TPO-binding sequence.

Glycosylation is not required for activity of the peptide-binding moiety of romiplostim, nor is it required for the Fc to bind to FcRn and thus, the Fc-peptide fusion protein as produced by the process of the present invention is non-glycosylated. Furthermore, glycosylated proteins exist, which are also active in their non-glycosylated form, for example human interferon-beta (Runkel et al., Pharm Res 15 (1998), 641-649). The absence of glycosylated forms are important advantages of the production in prokaryotic hosts, in particular in E. coli and thus, the present invention relates in a preferred embodiment to a process for the production of non-glycosylated proteins, in particular non-glycosylated Fc-peptide fusion proteins.

Structurally, romiplostim is a homodimer composed of two identical single chain subunits, each subunit consists of two tandem repeats of the TPO-binding peptide sequence separated by an eight-glycine linker and fused to the carboxy terminus of a human IgG1 Fc by another five-glycine linker (Shimamoto et al. 2012, supra). As an indispensable component of recombinant fusion proteins, like Fc-peptide fusion proteins, linkers have shown increasing importance in the construction of stable, bioactive fusion proteins. Suitable linkers to connect protein moieties and to connect proteins and peptides are known in the art; see for example Chen et al., Adv Drug Deliv Rev 65 (2013), 1357-1369. Accordingly, in one embodiment, the target peptide of the Fc-peptide fusion protein as produced by the process of the present invention is connected to the Fc moiety via a linker, which is preferably a linker composed of glycine residues, preferably an eight-glycine linker.

In a preferred embodiment, the Fc-peptide fusion protein as produced by the process of the present invention is romiplostim. The 14-amino acid binding domain of romiplostim was identified from screening of recombinant phage libraries of random peptides that stimulate TPO-dependent cell lines (Cwirla et al., Science 276 (1997), 1696-1699). The affinity of the peptide was optimized by mutagenesis and addition of flanking linkers. The peptide of romiplostim shares no sequence homology with endogenous TPO minimizing the development of cross-reacting antibodies. The amino acid sequence of romiplostim, in particular a monomer chain of romiplostim is set out in SEQ ID NO: 1.

Fc-peptide fusion proteins are valuable pharmaceuticals that can be used for the treatment of various diseases. Romiplostim was the first peptibody approved by both EMA and FDA, followed by dulaglutide, a glucagon-like peptide-1 (GLP-1) fused to an IgG4. The efficacy and safety of both peptibodies favored the investment of researchers to explore this strategy for the development of new therapeutic molecules. Thus, a considerable number of different peptibodies are currently in preclinical and clinical studies.

Accordingly, the present invention relates to a method of manufacturing a pharmaceutical composition comprising an Fc-peptide fusion protein, wherein the method comprises the steps of the process of the present invention for the production of the Fc-peptide fusion protein and a step of formulating the Fc-peptide fusion protein with a pharmaceutically acceptable carrier or buffer. Accordingly, the present invention also relates to a pharmaceutical composition produced by said method.

The pharmaceutical compositions of the present invention can be formulated according to methods well known in the art: see for example, Remington: The Science and Practice of Pharmacy (2000) by the University of Sciences in Philadelphia. ISBN 0-683-306472. Examples of suitable pharmaceutical carriers and buffers are well known in the art and include phosphate buffered saline solutions, water, emulsions, such as oil/water emulsions, various types of wetting agents, sterile solutions, buffer systems etc. Compositions comprising such carriers and buffers, respectively can be formulated by well-known conventional methods. These pharmaceutical compositions can be administered to the subject at a suitable dose. Administration of the suitable compositions may be effected by different ways, e.g., by intravenous, intraperitoneal, subcutaneous, intramuscular, intranasal, topical or intradermal administration or spinal or brain delivery. In a preferred embodiment, the pharmaceutical formulation is formulated for subcutaneous administration.

In a preferred embodiment, the Fc-peptide fusion protein, which is preferably romiplostim, is formulated with L-histidine, mannitol, polysorbate 20, sucrose and HCL. In one embodiment, mannitol can be substituted with sorbitol. In one embodiment, polysorbate 20 can be substituted with polysorbate 80. In one embodiment, mannitol can be substituted with sorbitol and polysorbate 20 can be substituted with polysorbate 80. Furthermore, in each of the three mentioned embodiments, HCl can be substituted with any other acid to adjust the pH to a target of about 5.

In one embodiment, the pharmaceutical composition of the present invention is present in a pharmaceutical container, preferably in a vial, more preferably a single-dose vial. Accordingly, the method of the present invention further comprises in one embodiment a step of filling the pharmaceutical composition in a pharmaceutical container, preferably in a vial, more preferably in a single-dose vial.

In one embodiment, the pharmaceutical composition is a powder, preferably a sterile, preservative-free, white powder. Accordingly, in one embodiment, the method of the present invention further comprises a step of lyophilizing the pharmaceutical composition.

The present invention further relates to the pharmaceutical composition produced by the method of the present invention, wherein each pharmaceutical container, preferably each vial comprises
(a) 230 mcg romiplostim, 0.7 mg L-histidine, 18 mg mannitol, 0.02 mg polysorbate 20.9 mg sucrose, and sufficient HCL to adjust the pH to a target of 5.0.
(b) 375 mcg romiplostim, 1.2 mg L-histidine, 30 mg mannitol, 0.03 mg polysorbate 20.15 mg sucrose, and sufficient HCl to adjust the pH to a target of 5.0; or
(c) 625 meg romiplostim, 1.9 mg L-histidine, 50 mg mannitol, 0.05 mg polysorbate 20.25 mg sucrose, and sufficient HCl to adjust the pH to a target of 5.0.

As explained hereinbefore and illustrated in the Examples, in principle host cells and expression control elements known in the art are adapted to and used in the novel production process of the present invention. Accordingly, the present invention extends the use of common recombinant expression systems to a process of large scale and high yield production of recombinant proteins under relatively mild conditions and thus with the benefit of saving energy, which in view of conducting the process in a bioreactor results in favorable economic and ecological balance compared to previous thermoregulated production processes. Therefore, the present invention also relates to the use of a recombinant prokaryotic host cell comprising a nucleic acid encoding an Fc-peptide fusion protein and which is operably linked to a temperature inducible promoter and/or a vector comprising the nucleic acid encoding an Fc-peptide fusion protein and which is operably linked to a temperature inducible promoter in the process of the present invention. In other words, the nucleic acid is under control of a temperature inducible expression system, which comprises a temperature sensitive repressor.

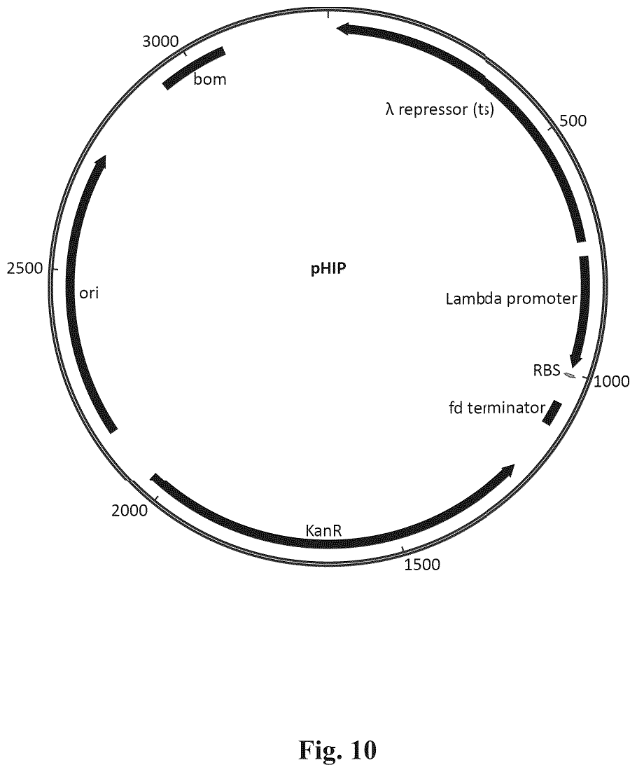

The vector which is used in accordance with the present invention comprises next to the nucleic acid encoding an Fc-peptide fusion protein, a temperature inducible promoter, which is preferably the lambda PR promoter, a temperature sensitive repressor, which is preferably the temperature sensitive lambda cI857 repressor, a kanamycin resistance gene, origin pBR322. ATP-E ribosomal binding site, and phage fd transcription terminator. The corresponding vector map is schematically depicted in FIG. 10. Of course, any kind of antibiotic resistance gene can be used which allows positive selection for host strains harboring the expression vector. Those include, but are not limited to genes conferring resistance to tetracycline, chloramphenicol, and ampicillin.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

The experiments performed in accordance with the present invention relate to the establishment of a scalable fed-batch fermentation process for the production of any kind of Fc-peptide fusion protein, exemplified with romiplostim.

### Material and Methods:

### Strain and Expression Vector

E. coli strain BL21 (F-ompT gal dcm lon hsdSB (rB-mB-) [malB+]K-12 (λS)) from Novagen® (Merck KGaA, Darmstadt, Germany) was transformed with the heat-inducible expression vector pHIP carrying the codon sequence optimized for the Fc-peptide fusion protein gene (romiplostim gene) under control of the lambda PR—cI857 system. Said vector is characterized by the following relevant features: Heat sensitive lambda cI857 repressor, kanamycin resistance, origin pBR322, lambda PR promoter, ATP-E ribosomal binding site, phage fd transcription terminator, and Fc-peptide fusion protein gene. The plasmid map of the pHIP-Fc-peptide fusion protein vector is shown in FIG. 10.

### Consumables

The consumables used for the experiments performed within the scope of the present invention are listed in Table 1.

TABLE 1
Consumables

| | | Manufacturer/ |
|---|---|---|
| Type | Name | Supplier |
| Inlet air filter for 10 L | Sartofluor junior | Sartorius Stedim |
| bioreactor Sartorius | cartridge | |
| Exhaust air filter for 10 L | Sartobran P mini | Sartorius Stedim |
| bioreactor Sartorius | cartridge - H9 | |
| Shake flask for pre- | Erlenmeyer flask with | Corning, Inc. |
| culture | vent cap, 0.5 and 2 L | |
| Single Use Bioreactor 1 L | BioBlue 1 f | Eppendorf AG |
| Filter for sterile | Acrodisc PF syringe | Pall Life Sciences |
| filtration of medium | filter 0.8/0.2 μm | |
| Filter for sterile | Bottle Top Filter | Fisher Scientific |
| filtration of medium | PES 0.22 μm | |
| Filter for sterile | Sartobran P Midicaps, | Sartorius Stedim |
| filtration of feed medium | 0.45-0.2 μm, 1 m² | |

### Media

Media compositions as well as media preparations used for the experiments performed within the scope of the present invention are described in detail in Tables 2 to G.

TABLE 2
Pre-culture medium KF1 preparation

| Application | Name | Composition | | | |
|---|---|---|---|---|---|
| (Process | (Preparation | | Conc. | MW | Weight-in |
| step) | record) | Substance | [mM/L] | [g/mol] | [g/L] |
| Pre-culture | Pre-culture | Glycerol | 271.47 | 92.09 | 25.0 |
| | medium KF1 | Yeast extract | n.a. | n.a. | 20.0 |
| | | L-Methionine | 13.40 | 149.21 | 2.0 |
| | | NaCl | 17.11 | 58.44 | 1.0 |
| | | KH₂PO₄ | 50.33 | 136.09 | 6.9 |
| | | K₂HPO₄ | 49.66 | 174.18 | 8.7 |
| | | Citric acid | 18.22 | 192.13 | 3.5 |
| | | MgSO₄ × 7H₂O | 8.11 | 246.48 | 2.0 |
| | | Thiamine HCl | 0.01 | 337.27 | 5 × 10⁻³ |
| | | | Conc. | MW | Weight-in |
| | | Substance | [mM/L] | [g/mol] | [mL/L] |
| | | Trace elements | n.a. | n.a. | 10.0 |
| | | solution | | | |
| | | (see Table 5) | | | |

Preparation/Storage
Preparation:
1. For a total volume of 1 L, all chemicals except MgSO₄ and thiamine
are dissolved in 950 mL DI—H₂O.
2. MgSO₄ and thiamine are dissolved in 40 mL DI—H₂O and added to
the medium after sterilization.
3. 10 mL of sterile trace elements solution are added to the medium
after sterilization.
pH and conductivity adjustment:
Target range pH: 7.0 ± 0.1
Sterilization:
20 min, ≥121° C. autoclaving except MgSO₄ and thiamine solution.
Filter sterilization with 0.2 μm syringe filters for MgSO₄ and thiamine
solution.
Storage:
3 months at room temperature after sterilization.

TABLE 3
Main fermentation medium KF1 preparation

| Application | Name | Composition | | | |
|---|---|---|---|---|---|
| (Process | (Preparation | | Conc. | MW | Weight-in |
| step) | record) | Substance | [mM/L] | [g/mol] | [g/L] |
| Main | Main | Glycerol | 54.29 | 92.09 | 5.0 |
| fermentation | fermentation | Yeast extract | n.a. | n.a. | 20.0 |
| | medium KF1 | L-Methionine | 13.40 | 149.21 | 2.0 |
| | | NaCl | 17.11 | 58.44 | 1.0 |
| | | KH₂PO₄ | 50.33 | 136.09 | 6.9 |
| | | K₂HPO₄ | 49.66 | 174.18 | 8.7 |
| | | Citric acid | 18.22 | 192.13 | 3.5 |
| | | MgSO₄ × 7H₂O | 8.11 | 246.48 | 2.0 |
| | | Thiamine HCl | 0.01 | 337.27 | 5 × 10⁻³ |
| | | Antifoam PPG | n.a. | n.a. | 0.2 |
| | | | Conc. | MW | Weight-in |
| | | Substance | [mM/L] | [g/mol] | [mL/L] |
| | | Trace elements | n.a. | n.a. | 10.0 |
| | | solution | | | |
| | | (see Table 5) | | | |

Preparation/Storage
Preparation:
1. For a total volume of 0.6 L, all chemicals except MgSO₄ and
thiamine are dissolved in 550 mL DI—H₂O.
2. MgSO₄ and thiamine are dissolved in 43.5 mL DI—H₂O and added
to the medium after sterilization.
3. 6.5 mL of sterile trace elements solution are added to the medium
after sterilization.
pH and conductivity adjustment:
Target range pH: 7.0 ± 0.1
Sterilization:
20 min, ≥121° C. autoclaving except MgSO₄ and thiamine solution.
Filter sterilization with 0.2 μm syringe filters for MgSO₄ and thiamine
solution.
Storage:
3 months at room temperature after sterilization.

TABLE 4
Kanamycin solution preparation

| Application | Name | Composition | | | |
|---|---|---|---|---|---|
| (Process | (Preparation | | Conc. | MW | Weight-in |
| step) | record) | Substance | [mM/L] | [g/mol] | [g/L] |
| Pre-culture | Kanamycin | Kanamycin | 25.75 | 582.58 | 15.0 |
| and main | solution | sulfate | | | |
| fermentation | | | | | |

Preparation/Storage
Preparation:
Kanamycin is dissolved in 10 mL DI—H₂O.
Sterilization:
Filter sterilization with 0.2 μm syringe filter.
Storage:
Immediate usage or 3 months at −20° C. after sterilization.

TABLE 5
Trace elements solution preparation

| Application | Name | Composition | | | |
|---|---|---|---|---|---|
| (Process | (Preparation | | Conc. | MW | Weight-in |
| step) | record) | Substance | [mM/L] | [g/mol] | [g/L] |
| Pre-culture | Trace elements | MnCl × 2H₂O | 10.13 | 161.88 | 1.64 |
| and main | solution | ZnCl₂ | 10.86 | 136.29 | 1.48 |
| fermentation | | CoCl₂ × 6H₂O | 1.83 | 147.85 | 0.27 |
| | | FeSO₄ × 7H₂O | 11.69 | 278.00 | 3.25 |
| | | CuSO₄ × 5H₂O | 0.88 | 249.68 | 0.22 |
| | | H₃BO₃ | 5.50 | 61.83 | 0.34 |
| | | Na₂MoO × 2H₂O | 1.31 | 205.92 | 0.27 |
| | | Titriplex ® III | 1.69 | 372.24 | 0.63 |
| | | | Conc. | MW | Weight-in |
| | | Substance | [mM/L] | [g/mol] | [mL/L] |
| | | HCl (10%) | n.a. | n.a. | 20 |

Preparation/Storage
Preparation:
All chemicals are dissolved in 500 mL DI—H₂O in the order given above.
Sterilization:
Filter sterilization with bottle top filter (0.2 μm).
Storage:
6 months after sterilization.

TABLE 6
Feed solution KF1.1 preparation

| Application | Name | Composition | | | |
|---|---|---|---|---|---|
| (Process | (Preparation | | Conc. | MW | Weight-in |
| step) | record) | Substance | [mM/L] | [g/mol] | [g/L] |
| Main | Feed solution | Glycerol | 3,257.68 | 92.09 | 300.0 |
| fermentation | KF1.1 | L-Methionine | 16.75 | 149.21 | 2.5 |
| fed batch | | Yeast extract | n.a. | n.a. | 150.0 |
| phase | | K₂HPO₄ | 30.43 | 174.18 | 5.3 |
| | | KH₂PO₄ | 14.70 | 136.09 | 2.0 |

Preparation/Storage
Preparation:
1. For 1 L of feed solution, glycerol and methionine are dissolved in
500 mL DI—H₂O.
2. Yeast extract, K₂HPO₄ and KH₂PO₄ are dissolved in 500 mL DI—H₂O.
3. Both solutions are mixed after sterilization.
Sterilization:
20 min, ≥121° C. autoclaving.
Storage:
6 months at room temperature after sterilization.

### Buffers

Buffers and corresponding buffer composition used for the experiments performed within the scope of the present invention are listed in Table 7.

TABLE 7
Buffers

| Process Step | Application | Composition |
|---|---|---|
| Main fermentation | pH control | 2M H₃PO₄ |
| | | 25% NH₄OH |
| | Antifoam agent | PPG 2,000 |
| | Sigma/A6426 | |
| CIP | Sanitization | 2M NaOH |
| | | 70% ethanol |
| Pre-culture and main | Buffer for OD | 0.9% NaCl |
| fermentation | measurement | |

### Equipment

Key equipment used for the experiments performed within the scope of the present invention is listed in Table 8.

TABLE 8
List of key equipment

| | Type/ | |
|---|---|---|
| Device | Manufacturer | Technical data |
| Bioreactor | DASGIP ® Bioblock/ | Total volume: 1.5 L |
| | Eppendorf | Working volume: 1 L |
| | | Vessel specifications: max. 0.5 bar |
| | | Vessel: single use |
| | | Stirring speed: 60-1,500 rpm |
| | | Max. power consumption stirrer: 20 W |
| | | Aeration rate: 0-250 sL/h (open pipe) |
| | | Acid-pump (peristaltic): 0-420 mL/h |
| | | Base-pump (peristaltic): 0-420 mL/h |
| | | Antifoam-pump (peristaltic): 0-420 mL/h |
| | Eppendorf | DO sensor (optic): 0-100% |
| | Mettler Toledo | pH sensor: pH 0-12 |
| | | (temperature compensated) |
| | DASGIP ® | Temperature sensor (Pt-100): −15 to 100° C. |
| Bioreactor | Biostat C/ | Total volume: 15 L |
| | Sartorius | Working volume: 10 L |
| | | Vessel specifications: −1/+3 bar; 150° C. |
| | | Height to diameter ratio: 3:1 |
| | | Vessel: stainless steel: 1.4435 |
| | | Surface roughness Ra: 0.8 μm (Rz = 5), |
| | | electro-polished |
| | | Stirring speed: 10-1,500 rpm |
| | | Max. power consumption stirrer: 900 W |
| | | Aeration rate: 0-30 sL/min (ring sparger) |
| | | Acid pump (peristaltic): 0.05-10 mL/min |
| | | (0.05 mL/min) |
| | | Base pump (peristaltic): 0.05-10 mL/min |
| | | (0.05 mL/min) |
| | | Antifoam pump (peristaltic): 0.05-10 mL/min |
| | | (0.05 mL/min) |
| | | Substrate pump (peristaltic): 0.05-10 mL/min |
| | | (0.05 mL/min) |
| | Mettler Toledo | DO sensor (optic): 0-100% |
| | | (temperature compensated) |
| | Mettler Toledo | pH sensor: pH 2-12 |
| | Sartorius | Temperature sensor (Pt-100): 0-150° C. |
| | Siemens | Pressure sensor: 0-2 bar |
| Cryostat | MC1200/ | Temperature range: −10 to 40° C. |
| | Lauda | Cooling capacity: 1200 W |
| Cryostat | FL1703/ | Temperature range: −10 to 40° C. |
| | Julabo | Cooling capacity: 2000 W |
| Autoclave | VX-150/ | — |
| | Systec | |
| Autoclave | DE23/ | — |
| | Systec | |
| Spectro- | Ultrospec 100 pro/ | Wavelength range: 190-1100 nm, in 0.1 nm steps |
| photometer | GE Healthcare | Wavelength accuracy: ±0.7 nm |
| | | Spectral bandwidth: <1.8 nm |
| Laminar flow | Hera Safe HSP 12/ | Circulating air flow: ~1100 m³/h |
| cabinet | Heraeus Instruments | Circulating air flow filter: ULPA |
| Centrifuge | Heraeus Fresco 21/ | Max. volume: 24 × 2 mL |
| | Thermo Scientific | Max. speed: 14,800 rpm |
| | | Max. force: 21,100 gₘₐₓ |
| | | Temperature range: −9° C. to 40° C. |
| Centrifuge | Sorvall RC 6+/ | Max. volume: 4 × 1.0 L |
| | Thermo Scientific | Max. Speed: 22,000 rpm |
| | | Max. Force: 55,200 gₘₐₓ |
| | | Temperature control: +2 to +40° C. |
| | | Temperature accuracy: ±2° C. |
| | | Bottle: PP |
| Centrifuge | Multifuge3 S-R/ | Max. volume: 4 × 0.9 L |
| | Heraeus | Max. Speed: 7,000 rpm |
| | | Max. Force: 9,200 gₘₐₓ |
| | | Temperature control: 2 to 7° C. at maximum |
| | | rotor speed |
| | | Temperature accuracy: ±2° C. |
| | | Bottle: PP |
| Incubator | Multitron Pro/ | Shaking amplitude: 25 mm |
| | Infors | Shaking frequency: 20-400 rpm |
| | | Temperature range: 20° C. under ambient |
| | | temperature up to 65° C. |
| | | Temperature accuracy: ±0.2° C. |
| Incubator | Ecotron/ | Shaking amplitude: 25 mm |
| | Infors | Shaking frequency: 20-400 rpm |
| | | Temperature range: 20° C. under ambient |
| | | temperature up to 65° C. |
| | | Temperature accuracy: ±0.2° C. |
| Freezer | FORMA 994/ | — |
| (−80° C.) | Thermo Scientific | |
| pH-Meter | MPC227/ | — |
| | Mettler Toledo | |
| Balance | BP6100/ | Weighing capacity: 0.01-6,200 g |
| | Sartorius | |
| Analytical | XSE 205DU/ | — |
| Balance | Mettler Toledo | |
| Oven | Modell 500/ | — |
| | Memmert | |
| Refrigerator | Premium/ | — |
| | Liebherr | |
| Analytical | Ultimate 3000/ | Max. pressure: 620 bar |
| UHPLC | Thermo Scientific | Max. flow rate: 10 mL/min |
| | | Min. cycle time: 15 s |
| | | Sample format: 384 or 96 Well plates, |
| | | vials up to 10 mL |
| | | Column: Hi-Plex H (300 × 7.7 mm; 14 mL) |
| | | Max. UV-vis detection rate: 100 Hz |
| Peristaltic | 323/ | — |
| pump | Watson Marlow | |

### Pre-Culture

Pre-culture runs were performed in 500 mL and 2 L shake flasks containing 75 and 300 mL of pre-culture medium (see Table 2). An inoculation ratio was chosen to ensure an OD₆₀₀ after inoculation in the shake flasks of 1.1×10⁻³. For the DoE phase and all following fermentation runs, 127 μL from the cell bank cryo culture vial were added to 300 mL of pre-culture medium. The pre-culture was incubated in a shaker incubator (details see Tables 8 and 9) at 30° C. and 220 rpm for 9 to 16 hours. At the end of incubation 50 mL of the pre-culture were used to inoculate the main fermenter.

TABLE 9
Process parameters for pre-culture fermentation

| Parameters/ | | |
|---|---|---|
| Equipment/Material | Condition | Unit |
| Medium | Pre-culture medium (see Table 2) | — |
| Temperature | 30 ± 1 | [° C.] |
| Agitation rate | 220 | [rpm] |
| Incubation time | 9-16 | [h] |
| OD₆₀₀ end of incubation | 1-2 | [—] |
| Transfer conditions | 50 mL are transferred to main fermenter | — |

### 1 L Main Fermentations

The 1 L main fermenter system used during the first development phase was DASGIP® multi fermenter system consisting of four 1 L polystyrene/polycarbonate single-use vessels, i.e. a bioreactor system (see Table 8 for details). Each vessel was filled with the main part of the sterile cultivation medium (for detailed media preparation, refer to Tables 3 and 5). The initial fermentation volume was 350 mL.

The sterility of the medium was controlled by running a sterile-test for at least 8 hours under process conditions without pH-control prior to fermenter inoculation. The fermenter was considered sterile when the pH had a maximum variation within +0.2 pH units. Additionally, stable values for the pO₂ and the appearance of clear medium were considered indications for a sterile medium.

The Fc-peptide fusion protein production fermentation was performed in fed-batch mode. The respective process conditions are listed in Tables 10 and 11. The main fermenter was inoculated with 50 ml of cell broth taken from the pre-culture fermentation. During the process, the dissolved oxygen tension was kept at 30% applying a control cascade which acts first on stirrer rate and then on enrichment of the inlet air flow with oxygen. When the carbon source was depleted, a sharp increase of the DO-signal triggered automatically the start of the feed solution addition with an exponential feeding profile. The profile was calculated with equation (1). The point of induction was varied during development (OD₆₀₀=30/80/140±5). The culture was induced by increasing the temperature of the vessels to 39-42° C. The duration of the expression phase was varied in between 6 and 28 h. For the temperature changes, a uniform distribution is achieved by a so-called disk blade stirrer, on which three disk blade elements are arranged at the bottom, in the middle and at the top of the fermenter. For the measurement of the temperature a standard PT100 element is used, which has a measuring point in the medium.

TABLE 10
Process parameter for 1 L scale fermentations

| Parameter | Condition | Unit |
|---|---|---|
| Medium | Main fermentation medium | — |
| | (Table 3) | |
| Adjusting solutions | NH₄OH (25%); H₃PO₄ (2M) | — |
| | Antifoam PPG 2,000 | |
| Start volume in fermenter | 350 | [mL] |
| Inoculation volume | 50 | [mL] |
| Induction OD₆₀₀ | 30/80/140 | — |
| Induction temperature | 39/40/42 | [° C.] |
| Expression phase | 6-28 | [h] |
| Overall process time main | 17-34 | [h] |
| fermentation | | |

TABLE 11
Control loops for 1 L scale fermentations

| Control Loops | Value | Unit |
|---|---|---|
| Cultivation temperature | 30 ± 1 | [° C.] |
| pH (acid/base addition) | 7.0 ± 0.1 | [—] |
| pO_{2w} (pO₂ cascade control | 30 | [%] |
| agitation and oxygen enrichment) | | |
| Initial stirring rate | 300 | [rpm] |
| Max. stirring rate | 1,500 | [rpm] |
| Air flow (mass flow controller) | 1 | [vvm] |

### 10 L. Main Fermentations

The main fermenter system used during the second development phase was a Sartorius Biostat C with 10 L working volume. The bioreactor was filled with the main part of the cultivation medium and was sterilized in-situ. Afterwards, the medium was completed to 6.5/7.8/8.0 L total fermentation volume.

The sterility of the medium was controlled by running a sterile-test for at least 8 hours under process conditions without pH-control prior to fermenter inoculation. The fermenter was considered sterile when the pH had a maximum variation within ±0.2 pH units. Additionally, stable values for the pO₂ and the appearance of clear medium were considered indications for a sterile medium.

The Fc-peptide fusion protein production fermentation was performed in fed-batch mode. The respective process conditions are listed in Tables 12 and 13. The main fermenter was inoculated with 200/240/246 mL of cell broth taken from the pre-culture. During the process, the dissolved oxygen tension was kept at 30% applying a control cascade which acts first on stirrer rate and then on enrichment of the inlet air flow with oxygen. In fermentation run U75 this control cascade was changed, so that instead of enriching the inlet air with oxygen, the tank pressure was increased from 0.0 to 0.8 bar. When the carbon source was depleted, a sharp increase of the DO-signal triggered automatically the start of the feed solution addition with an exponential feeding profile (see equation 1). The culture was induced at an OD₆₀₀ of 80±5 by increasing the temperature of the vessel to 39° C. The expression phase lasted 5-10 h. For the temperature change, a uniform distribution is achieved by a so-called disk blade stirrer, on which three disk blade elements are arranged at the bottom, in the middle and at the top of the fermenter. For the measurement of the temperature a standard PT100 element is used, which has a measuring point in the medium.

$$F = \frac{{μ}_{w} * {V}_{L 0} * {c}_{XL}}{{Y}_{X / S} * {c}_{S}} * {e}^{( μ * ( t - {t}_{0} ) )} \qquad ( 1 )$$

F=feed flow rate [mL/h]
μ_{w}=set point for growth rate [h-1]
V_{L0}=liquid reactor volume at feeding start [mL]
C_{XL}=cell dry weight at feeding start [g/mL]
Y_{X/S}=yield coefficient substrate/biomass [g/g]
cₛ=concentration of carbon source in feeding solution [g/mL]
t=process time [h]
t₀=time at feed start [h]

TABLE 12
Process parameters for 10 L main fermentation

| Parameter | Condition | Unit |
|---|---|---|
| Medium | Main fermentation medium | — |
| | (Table 3) | |
| Adjusting solutions | NH₄OH (25%); H₃PO₄ (2M); | — |
| | Antifoam PPG 2,000 | |
| Start volume in fermenter | 6.5/7.8/8.0 | [L] |
| Inoculation ratio | 3.1 | [% (v/v)] |
| Induction OD₆₀₀ | 80 | — |
| Induction temperature | 39 | [° C.] |
| Expression phase | 6-10 | [h] |
| Overall process time main | 17-28 | [h] |
| fermentation | | |
| Heating rate | usually 3.6 | [° C./min] |

TABLE 13
Control loops for 10 L main fermentations

| Control Loops | Value | Unit |
|---|---|---|
| Cultivation temperature | 30 ± 1 | [° C.] |
| pH (acid/base addition) | 7.0 ± 0.1 | [—] |
| pO_{2w} (pO₂ cascade control | 30 | [%] |
| agitation and oxygen enrichment) | | |
| Initial stirring rate | 300 | [rpm] |
| Max. stirring rate | 1,500 | [rpm] |
| Air flow (mass flow controller) | 1 | [vvm] |
| Max. tank pressure | 800 | [mbar] |

### Harvest

Cell harvest was conducted via centrifugation for 20 min at 9,200 g and 4° C. Afterwards, the supernatant was separated from the cell pellet and the latter was stored at −20° C. for further DSP processing.

### Analytical Methods

Different online parameters were monitored during the fermentation run (refer to Table 14). Offline parameters (listed in Table 15) were analyzed subsequent to the fermentation. During the fermentation, the OD₆₀₀ was measured at-line directly after sampling (Ultrospec 3300 pro Spectrometer, GE Healthcare). 0.9% NaCl was used as blank and diluent. The cell dry weight was determined after drying a cell pellet of 1 mL for 24 h at 80° C. Fc-peptide fusion protein product yield was determined based on SDS-PAGE analytics with reference protein and subsequent densitometric analysis via the software Phoretix One® 1-D. Analytics of residual glucose, glycerol and acetate in the medium were conducted with HPLC analysis using an Ultimate 3000 UHPLC by Thermo Fisher Scientific.

TABLE 14
Monitored online parameters

| | |
|---|---|
| DASGIP ® | Sartorius Biostat C |
| (1 L scale) | (10 L scale) |

| | |
|---|---|
| Online parameters | Temperature ϑ_{L} |
| monitored with the process | pH value |
| control systems | H₃PO₄ volume |
| | NH₄OH volume |
| | Antifoam (PPG) volume |
| | Feeding volume |
| | Stirring rate N_{St} |
| | Partial pressure of dissolved oxygen pO₂ |
| | Aeration rate (air/O₂ gasmix) |
| | Pressure in the vessel (Biostat C only) |

TABLE 15
Overview on analytical methods

| Parameter | Method |
|---|---|
| Optical Density | Extinction at 600 nm |
| CWW/CDW | Gravimetric |
| Product content | SDS-PAGE |
| Concentration of glycerol, | HPLC |
| glucose and acetate in the | |
| fermentation medium | |
| Growth rate | Theoretical value based on the |
| | settings of the feed, i.e. the |
| | feeding rate follows an |
| | exponentially increasing profile |
| | (see equation 1) in order to |
| | maintain a constant specific |
| | growth rate |

### Example 1: A Mild Temperature Shift and an Induction OD₆₀₀≥30 Leads to High Fc-Peptide Fusion Protein Production in 1 L Scale Fermentation Runs

Important process parameters were screened by the means of design of experiments (DoE) in 1 L scale to develop an upstream process with maximum Fc-peptide fusion protein titer at the end of fermentation (EoF). For a detailed process description of the 1 L fermentation runs; see “Materials and Methods”, above. Fermentation parameters and control loops are listed in Table 9 (pre-culture) as well as in Tables 10 and 11 (main fermentation). The utilized glycerol containing KF1 pre-culture, culture and feed media are described in the “Materials and Methods” section in Tables 2, 3 and 6, respectively.

### Experimental Setup

For cultivation experiments, the E. coli strain harboring the Fc-peptide fusion protein expression vector as defined in the “Material and Method” section was used. Furthermore, a total expression time of 12 h was defined for all DoE fermentation runs. The following parameters were identified to have potential impact on product formation and were therefore tested in specific ranges (see Table 16). Induction temperature was tested within the range of 39° C. to 42° C. A lower induction temperature than 39° C. was considered not feasible due to limitations of the expression system and was therefore not tested.

TABLE 16
Process parameter variation for DoE

| Parameter | Range |
|---|---|
| Growth rate: μ [1/h] | 0.1-0.2 |
| Induction OD: OD_{Ind} [—] | 30-80 |
| Induction temperature: T_{Ind} [° C.] | 39-42 |
| Concentration of methionine in feed | 0-6 |
| solution: cₘₑₜₕ [g/L] | |

The DoE target parameter was the maximum Fc-peptide fusion protein concentration (cPIL, max) achieved during the fermentation. The corresponding productivity (Prmax, maximum Fc-peptide fusion protein concentration per fermentation time at maximum concentration) was calculated and taken into account for the overall assessment of the process strategy. The parameter set points for the DoE fermentations including the results U48-U63 are listed in Table 17.

TABLE 17
Process parameter set points for DoE and results

| | μ | OD_{Ind} | T_{Ind} | cₘₑₜₕ | c_{P1L, max} | Prₘₐₓ | tₘₐₓ |
|---|---|---|---|---|---|---|---|
| Run | [1/h] | [—] | [° C.] | [g/L] | [g/L] | [g/L h] | [h] |
| U48 | 0.1 | 30 | 39 | 0 | 3.8 | 0.189 | 20.1 |
| U49 | 0.1 | 30 | 42 | 0 | 1.5 | 0.075 | 19.9 |
| U50 | 0.1 | 30 | 39 | 6 | 3.4 | 0.188 | 18.1 |
| U51 | 0.1 | 30 | 42 | 6 | 1.9 | 0.095 | 20.1 |
| U52 | 0.2 | 30 | 39 | 0 | 2.3 | 0.131 | 17.6 |
| U53 | 0.2 | 30 | 42 | 0 | 1.8 | 0.102 | 17.6 |
| U54 | 0.2 | 30 | 39 | 6 | 2.7 | 0.153 | 17.6 |
| U55 | 0.2 | 30 | 42 | 6 | 2.3 | 0.131 | 17.6 |
| U56 | 0.1 | 80 | 39 | 0 | 10.5 | 0.352 | 30.4 |
| U57 | 0.1 | 80 | 42 | 0 | 4.5 | 0.149 | 30.2 |
| U58 | 0.1 | 80 | 39 | 6 | 10.7 | 0.377 | 28.4 |
| U59 | 0.1 | 80 | 42 | 6 | 4.7 | 0.156 | 30.2 |
| U60 | 0.2 | 80 | 39 | 0 | 10.0 | 0.578 | 17.3 |
| U61 | 0.2 | 80 | 42 | 0 | 4.6 | 0.217 | 21.2 |
| U62 | 0.2 | 80 | 39 | 6 | 10.2 | 0.590 | 17.3 |
| U63 | 0.2 | 80 | 42 | 6 | 1.7 | 0.088 | 19.3 |

### Results and Conclusion

The maximum Fc-peptide fusion protein yield achieved during the fermentations is shown in Table 17. The fermentation runs with an induction OD₆₀₀ of 30 (U48-U55) resulted in a significantly lower maximum product concentration than the runs with an induction OD₆₀₀ of 80 (US6-U63). Moreover, the fermentations with a lower induction temperature of 39° C. (even numbers) resulted in a higher Fc-peptide fusion protein concentration than those with an induction temperature of 42° C. The combination of both factors resulted in the highest concentrations of about 10 g/L (U56, U58, U60 and U62, refer to Table 17). The productivity calculated for the time point of maximum Fc-peptide fusion protein concentration is listed in Table 17.

A high induction OD₆₀₀ and a lower induction temperature result in higher productivity. However, the productivity indicates the influence of a third factor. Relating to maximum product concentration, the four fermentation runs show comparable results. However, the productivity of run U56 and U58 is significantly lower than the productivity of the runs U60 and U62 (0.35 and 0.38 g/Lh compared to 0.58 and 0.59 g/Lh). The fermentation runs U56 and U58 were conducted with a growth rate of 0.1 l/h while U60 and U62 were conducted with a growth rate of 0.2 l/h. Therefore, a combination of a high induction OD₆₀₀, a low induction temperature and a growth rate of 0.2 l/h results in a high productivity. Thus, high Fc-peptide fusion protein yields can be achieved in a shorter process time than with a growth rate of 0.1 l/h. The difference in the product concentration course regarding the growth rate is also shown in FIG. 2.

FIG. 2 shows the course of the Fe-peptide fusion protein concentration of the fermentation runs U56 and U60. The Fc-peptide fusion protein concentration in run U56 increased in the first 8 h of expression time to 10.6 g/L, before decreasing to 8.8 g/L after 10 h of expression time. In the last two hours of the process the product concentration increased again to a final 10.7 g/L. The Fc-peptide fusion protein concentration of fermentation run U60 had already reached its maximum of 10.0 g/L after 4 h of expression time. Afterwards, the concentration decreased to a value of 5.6 g/L at the end of the fermentation. This comparison demonstrates that the growth rate solely affects the productivity yet not the maximum Fc-peptide fusion protein concentration. Therefore, a fermentation strategy with a higher growth rate of 0.2 l/h could potentially result in high product titers in a shorter process time.

Of the four parameters tested the induction OD₆₀₀ and the induction temperature displayed the most significant influence on Fc-peptide fusion protein yield. The results of the DoE experiments revealed the benefit of a high induction OD₆₀₀ of 80 combined with a low induction temperature of 39° C. resulting in high Fc-peptide fusion protein yields, with product values of up to 10 g/L. Furthermore, the applied growth rates only affected productivity, given that comparable maximum Fc-peptide fusion protein titers were reached with both the growth rate 0.1 l/h and 0.2 l/h, yet in a significantly reduced processing time (8 h shorter) with a growth rate of 0.2 l/h. Given that the methionine in the feed medium did not have any impact on product yield, it was left out of the feed solution formulation for the following experiments. Since the product concentration in fermentation runs with a growth rate of 0.2 l/h began to decrease after 6-8 h of expression time, an expression time of 6 h was defined as point of EoF.

In summary, a process strategy with an induction OD₆₀₀ of 80, an induction temperature of 39° C., a growth rate of 0.2 l/h, 6 h expression time and KF1 pre-culture, culture, and feed medium was therefore implemented, whereby methionine was left out of the KF1 feed medium.

### Example 2: An Induction OD₆₀₀>80 Leads to a Further Improved Fc-Peptide Fusion Protein Production and an Impact of Kanamycin on Cell Growth and Fc-Peptide Fusion Protein Formation in 1 L Scale Fermentation Runs was not Observed

Further small scale (SmSc) experiments were conducted to gain more data about the process and the impact of parameter variations on growth and Fc-peptide fusion protein formation. For a detailed process description of the 1 L fermentation runs, see “Materials and Methods”, above. Fermentation parameters and control loops are listed in Table 9 (pre-culture) as well as in Tables 10 and 11 (main fermentation). The utilized glycerol containing KF1 pre-culture, culture and feed media are described in the “Materials and Methods” section in Tables 2, 3 and 6, respectively.

### Experimental Setup

The DoE fermentation runs identified the induction OD₆₀₀ and the induction temperature as the two key factors to play a significant role in product formation. Given that a lower induction temperature than 39° C. was considered as not feasible due to limitations of the expression system, only the impact of a further increased induction OD₆₀₀ on growth and product formation was tested in the fermentation runs U69 (induction OD₆₀₀ of 93), U70 (induction OD₆₀₀ of 100) and U71 (induction OD₆₀₀ of 140), respectively. Furthermore, the fermentation runs U69 and U70 were conducted to examine a potential effect of kanamycin added to the fermentation medium on product formation.

### Results and Conclusion

The comparison of the fermentation runs U70 with and U69 without kanamycin are shown in FIG. 3. The OD_{Ind} for these fermentations was 93 and 100 respectively. The OD₆₀₀ of both fermentations increased within the first 17 h to 212 and 228 respectively. Afterwards, the OD₆₀₀ of fermentation run U69 decreased to a temporary minimum of 183 four hours later before increasing again to the final and maximum value of 230. In contrast, the OD₆₀₀ of run U70 had already reached its maximum after 17 h of fermentation time and decreased continuously afterwards, ending at a value of 137. The Fc-peptide fusion protein concentration of run U69 increased rapidly, reaching a maximum of 14.3 g/L after 2 h of expression time. Subsequently, the concentration decreased almost in the same velocity as it increased before, reaching a value of 3.2 g/L at EoF. After 4.3 h of expression time, the product concentration of run U70 had already reached its possible maximum of 14.3 g/L. Data from before this time point are not available and hence the likelihood of maximum product titers at earlier process stages cannot be evaluated. In the following course of the fermentation, the Fc-peptide fusion protein concentration of run U70 decreased, reaching a final value of 7.1 g/L. The curves of the optical density of these two runs are divergent in the last hours of the fermentation. However, regarding the potential EoF time point of 6 h expression time, the OD₆₀₀ courses appear to be similar. The courses and maximum values of the product concentrations were considered comparable in respect of reaching high concentrations after a short process time and afterwards decreasing continuously. An impact of kanamycin in the fermentation medium on product formation within the specified 6 h of expression time was therefore not observed. Thus, kanamycin can be left out of fermentation medium formulation.

Compared to the results of DoE run U60, the maximum achieved Fe-peptide fusion protein concentration of the runs U69 and U70 induced at an OD₆₀₀ of 95±5 was about 40% higher than run U60 which was induced at an OD₆₀₀ of 80.

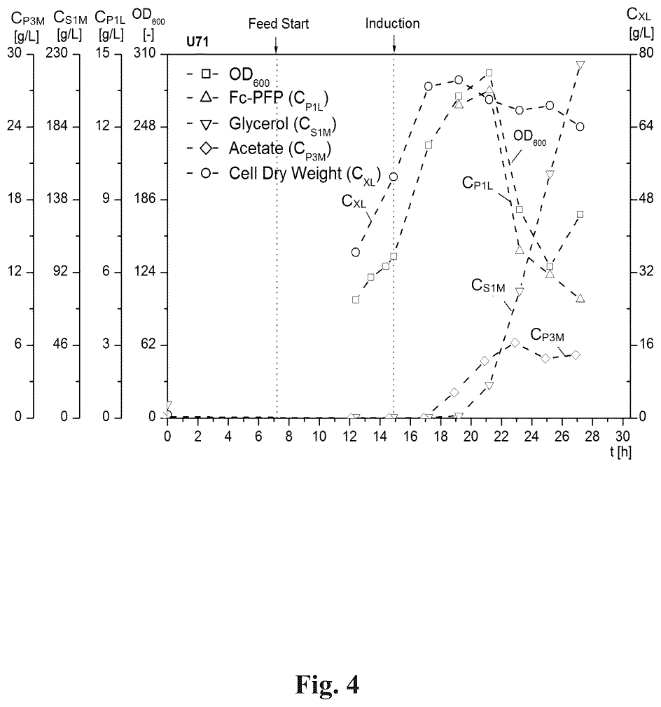

FIG. 4 depicts the data obtained from offline sample analytics for the fermentation run U71 where expression was induced at an induction OD₆₀₀ of 140. The OD₆₀₀ increased within 21 h of fermentation time to a maximum value of 294. After having reached this maximum, the OD₆₀₀ decreased rapidly in the following 4 h to a temporary minimum of 129 before increasing again in the last two hours to 174 at EoF. The product concentration followed a very similar course, reaching its maximum as well after 21 h of fermentation time at 13.5 g/L. Subsequently, the product concentration decreased rapidly to a final value of 4.9 g/L at EoF. Acetate began to accumulate in the medium after 19 h of fermentation time, reaching a maximum value of 6.2 g/L after 23 h. With only a short delay, glycerol started accumulating in the medium as well. The concentration of glycerol increased constantly to a maximum value of 223.8 g/L at the end of the fermentation. This accumulation of glycerol indicates that the cells had stopped its metabolization which matches the decrease of OD₆₀₀ and the cell dry weight (CDW) at around the same time due to lack of cell growth. The maximum concentration of Fc-peptide fusion protein in fermentation run U71 was about 35% higher than in fermentation run U60. Furthermore, the higher induction OD₆₀₀ seemed to have a major effect on maximum OD₆₀₀, given that it reached an all-time high value of 294, which was 30% higher than the maximum value of 226 achieved in run U60.

In conclusion, the fermentations U69 to U71 with product formation induced at OD₆₀₀ of 93, 100 and 140 respectively, achieved 35 to 40% higher maximum Fc-peptide fusion protein yields than fermentation run U60 which was induced at an OD₆₀₀ of 80. However, accelerated product degradation was also observed. Evidently and with regard to production levels, an induction OD₆₀₀ of approximately 100 was beneficial, given that the maximum Fc-peptide fusion protein yield of run U71 (induction OD₆₀₀=140) was 6% lower than the Fc-peptide fusion protein concentration in the runs U69 and U70.

In all three fermentations, the product concentration decreased rapidly after having reached its maximum. It was therefore considered likely that the point of maximum Fc-peptide fusion protein concentration would be missed in a process set-up with EoF based on the parameters OD₆₀₀ or fermentation time. To ensure a maximum Fc-peptide fusion protein yield at EoF and a robust process design regarding its time point, the induction OD₆₀₀ was kept at 80 for the following fermentations. Furthermore, a process strategy with a growth rate of μ=0.2 l/h and an induction temperature of about 39° C. was implemented.

### Example 3: A Dissolved Oxygen (DO) Concentration of about 30% was Found to be Optimal for Cell Growth and Fc-Peptide Fusion Protein Yield in 1 L Scale Fermentation Runs

In the fermentation runs U76-U78, the set point of the DO was varied to investigate possible negative effects on cell growth and product formation. For a detailed process description of the 1 L fermentation runs, see “Materials and Methods”, above. Fermentation parameters and control loops are listed in Table 9 (pre-culture) as well as in Tables 10 and 11 (main fermentation). The utilized glycerol containing KF1 pre-culture, culture and feed media are described in the “Materials and Methods” section in Tables 2, 3 and 6, respectively.

### Experimental Setup

In the fermentation runs U76-U78, the set point of the DO was decreased to 24%, 16%, and 8%, respectively, to investigate potential impacts on cell growth and product formation. According to the conclusion drawn from Example 2, a process strategy with a growth rate of μ =0.2 l/h, an induction OD₆₀₀ of 80, and an induction temperature of 39° C. was applied.

### Results and Conclusion

Cell growth and Fc-peptide fusion protein concentrations achieved in fermentation runs U76-U78 with lower DO set points compared to those of a run with a set point of 30% are listed in Table 18.

TABLE 18
Results of the DO set point comparison

| Parameter | U60 | U76 | U77 | U60 | U76 | U77 | U78 |
|---|---|---|---|---|---|---|---|
| DO [%] | 30 | 24 | 16 | 30 | 24 | 16 | 8 |
| Fc-peptide | 10.1 | 8.4 | 7.8 | n.d. | 4.7 | 4.3 | 4.7 |
| fusion protein | | | | | | | |
| [g/L] | | | | | | | |
| OD₆₀₀ [—] | 225 | 208 | 193 | 156 | 134 | 143 | 133 |
| tₑₓₚᵣ [h] | 6 | 6 | 6 | 2 | 1.5 | 1.6 | 1.5 |

A lower DO set point seems to have an effect on cell growth, given that the OD₆₀₀ of run U60 with a set point of 30% after 2 h of expression was slightly higher than in the runs U76-U78 and after 6 h it was significantly higher than in the runs U76-U78. Furthermore, the Fc-peptide fusion protein concentrations of the runs U76 and U77 after 6 h of expression time are about 20% lower than the concentration in run U60. This could indicate that reducing the DO set point from 30 to 8% has a negative impact on cell growth and product formation. Thus, the DO set point was maintained at 30% and was subsequently considered a critical process parameter. The DO should be monitored and should not drop below 10% for longer than 1 h to avoid negative influences on product yield or cell growth.

### Example 4: Scale Up to 10 L Fermentation Volume was Successful

The developed process strategy was scaled up to 10 L scale in a bioreactor. For a detailed process description of the 10 L fermentation runs see “Materials and Methods”, above. Fermentation parameters and control loops are listed in Table 12 and in Table 13, respectively. The utilized glycerol containing KF1 pre-culture, culture and feed media are described in the “Materials and Methods” section in Tables 2, 3 and 6, respectively.

### Experimental Setup

Based on the results of the first SmSc development phase the process strategy according to reference run U60 (see Table 17, FIG. 2) with induction OD₆₀₀ (OD_{Ind})=80, induction Temperature (Tind)=39° C., growth rate (μ)=0.2 l/h. Methionine concentration in feed solution (cMeth)=0 g/L, expression time (texpr)=6 h, KF1 pre-culture, culture and feed medium, was scaled up to 10 L scale.

### Results and Conclusion

FIG. 5 shows the offline sample analysis for exemplary fermentation run U92. Glycerol concentration decreased in the first 8.7 h from an initial 7.8 g/L as glycerol was consumed by the cells. The depletion of glycerol in the fermentation medium marked the end of the batch phase and the start of the feed. Acetate started accumulating in the medium after 17 h of fermentation time. The maximum concentration of 0.8 g/L was reached after 23.2 h of fermentation time, reaching a maximum of 5.8 g/L at EoF. As glycerol was consumed, the OD₆₀₀ started to increase and reached a maximum value of 205 after 17 h of fermentation time. Subsequently, the OD₆₀₀ decreased slightly to a final value of 191 at the end of the fermentation. The cell dry weight (CDW) increased simultaneously with the OD₆₀₀ ending up at a final value of 55.4 g/L. The concentration of the Fc-peptide fusion protein increased rapidly after induction and added up to 11.5 g/L after 17 h. In the last two hours of the fermentation, the product concentration remained more or less constant ending at 11.6 g/L.

In conclusion, the scale up to 10 L was considered successful. The fermentations showed similar or even higher maximum Fc-peptide fusion protein concentrations compared to the reference run U60 in 1 L scale. The Fc-peptide fusion protein concentration did not rapidly decrease as it probably would have had with a higher induction OD₆₀₀. Furthermore, reproduction of the process strategy revealed no technical issues in 10 L scale.

In further fermentation runs, a potential impact of the feed solution sterilization procedure was investigated. Accordingly, two bioreactor cultivations were performed, wherein in one fermentation run (U72), the feed solution was sterilized in an autoclave (20 min at 121° C.) as it was done in all previous fermentations, whereas in another fermentation run (U73), the feed solution was sterilized by filtration (0.2 μm; see Table 1).

The OD₆₀₀ of both fermentations increased simultaneously up to values of 206 and 198 respectively after 13 h of fermentation time. Subsequently, the OD₆₀₀ of run U72 stayed constant for two hours before decreasing to a final value of 199. The OD₆₀₀ of run U73 increased in the following 4 h to a final value of 224. The Fc-peptide fusion protein concentration of both fermentations increased simultaneously up to a maximum value of 10.4 and 10.9 g/L respectively after 4 h of expression. Afterwards, the concentration decreased in run U72 to a final value of 8.6 g/L and in run U73 to a slightly higher 10.1 g/L. Both runs showed very comparable courses of DO. OD₆₀₀, and Fc-peptide fusion protein concentration. Run U73 with the filter sterilized feed solution achieved a 13% higher OD₆₀₀ and a 17% higher Fc-peptide fusion protein concentration. The sterilization via filter was therefore considered to slightly improve growth and product formation. Thus, this sterilization procedure for the feed solution was implemented for all subsequent fermentations.

### Example 5: A Pressure DO-Control Cascade was Implemented for 10 L Scale Fermentation Runs

To establish a process which would be technically feasible in the production site, further adaptations of the process strategy were implemented. In particular, the DO-control cascade was modified and the suitability of a DO-cascade with pressure as second actor was tested.

For a detailed process description of the 10 L fermentation runs see “Materials and Methods”, above. Fermentation parameters and control loops are listed in Table 12 and in Table 13, respectively. The utilized glycerol containing KF1 pre-culture, culture and feed media are described in the “Materials and Methods” section in Tables 2, 3 and 6, respectively.

### Experimental Setup

Instead of increasing the amount of oxygen in the inlet air after the stirrer rate had reached its maximum, the pressure in the fermentation reactor was now increased from 0.0 to 0.8 bar(g) to assure a stable DO value of 30% throughout the fermentation.

To achieve reasonable durations of expression the growth rate had to be reduced as well. In the fermentations with a growth rate of 0.2 l/h a high demand of oxygen by the cells was observed. An increase in pressure to 0.8 bar(g) could not provide the required amount of dissolved oxygen in the medium. To ensure slower oxygen consumption by the cells, the growth rate was decreased to 0.1 l/h. Given that the fermentation runs with this growth rate in the DoE (refer to run U56 and U58. Table 17) achieved comparable maximum Fc-peptide fusion protein concentration albeit after longer process times, the reduction of growth rate was considered feasible with good Fc-peptide fusion protein yields.

These adaptations were tested in fermentation run U75, which was otherwise conducted under the same conditions as outlined in Example 4. i.e., with induction OD₆₀₀ (OD_{Ind})=80, induction Temperature (Tind)=39° C., methionine concentration in feed solution (cMeth)=0 g/L, expression time (texpr)=6 h, KF1 medium.

### Results and Conclusion

The data of the implementation run U75 is shown in FIG. 6. After 19.5 h of fermentation time, the stirrer rate had reached its maximum. Subsequently, the pressure in the vessel started to increase automatically from 0.0 to 0.8 bar(g). After 23.8 h the pressure had also reached its maximum value. To prolong the fermentation the inlet air flow rate was then manually increased from an initial 7.8 to a maximum 11.8 L/min (which corresponds to 1-1.5 vvm, respectively). Thereby it was possible to extend the fermentation by two hours, reaching a total of 9 h of expression time. The OD₆₀₀ increased continuously during the fermentation, reaching a final value of 196 after 26 h of fermentation time. Also, the Fc-peptide fusion protein concentration increased continuously, adding up to a maximum value of 7.4 g/L (based on SDS-PAGE quantification, Phoretix software).

The fermentation run U75 with the implemented pressure DO-cascade was considered technically feasible and therefore successful despite a 25% reduction in Fc-peptide fusion protein concentration and an extended process time (11 h longer) compared to the previous scale up runs.

Due to the necessity to establish a feasible process for the production site, the pressure DO-cascade was implemented for all subsequent fermentations. Furthermore, the inlet air flow was increased to constant 1.5 vvm and the growth rate was decreased to 0.1 l/h.

### Example 6: The Induction Temperature Showed a Significant Influence on Fc-Peptide Fusion Protein Production in 10 L Scale Fermentations, Whereas the Process was Robust Regarding Process Parameter Variations of Induction OD₆₀₀, Feed Flow Rate and pH

A robustness testing was performed to investigate the possible impacts of potential process related variations. For a detailed process description of the 10 L fermentation runs see “Materials and Methods”, above. Fermentation parameters and control loops are listed in Table 12 and in Table 13, respectively. The utilized glycerol containing KF1 pre-culture, culture and feed media are described in the “Materials and Methods” section in Tables 2, 3 and 6, respectively.

### Experimental Setup

With the new adaptations pressure DO-cascade, constant inlet air flow 1.5 vvm and growth rate of 0.1 l/h, robustness assessment was performed in fermentation runs U79 to U90 via DoE to investigate the possible impacts of potential process related variations on Fc-peptide fusion protein yield.

Four factors were identified as important process factors with potential influence on overall yield when deviating from its optimum set point: induction OD₆₀₀, feed flow rate (FFeed), pH and induction temperature (Tind) (Table 19). The DoE was conducted with the variation of these four factors to investigate whether the deviation of one or more factors has a statistically significant influence on product yield.

TABLE 19
Parameter variation for robustness DoE

| Parameter | Range |
|---|---|
| Induction OD: OD_{Ind} [—] | 65-95 |
| Feed rate deviation: F_{Feed} [%] | 90-110 |
| pH during the fermentation: pH [—] | 6.5-7.5 |
| Induction temperature: T_{Ind} [° C.] | 38-40 |

A fractional factorial design (resolution IV) with three center point (CP) fermentation runs was conducted for robustness testing. In a fractional factorial design, the number of experiments are defined according to the formula 2^{k-1} with k=numbers of parameters (factors) identified as important process factors and-1 as set for fractional factorial design (Example 6:4 Parameters, -1=3→2³=8 fermentation runs and 3 CPs). The fractional factorial design (resolution IV) with three center points (CP) was automatically generated with the software MODDE from Umetrics/Sartorius Stedim (Eriksson et al., Design of Experiments, Principles and Applications 3: Stockholm (2008), ISBN 9789197373043 9197373044, 459 pp).

The process parameter values for the three CP fermentation runs U79, U81 and U82 were set as the mean value of the upper and the lower range limit. The DoE target parameter was the Fc-peptide fusion protein concentration at EoF (cPIL). The parameter set points for all 11 DoE fermentations including the results are listed in Table 20.

TABLE 20
Parameter set points for robustness DoE and results

| | OD_{Ind} | F_{Feed} | pH | T_{Ind} | c_{P1L} |
|---|---|---|---|---|---|
| Run | [—] | [%] | [—] | [° C.] | [g/L] |
| U79 | 80 | 100 | 7.0 | 39 | 4.8 |
| U81 | 80 | 100 | 7.0 | 39 | 6.4 |
| U82 | 80 | 100 | 7.0 | 39 | 5.3 |
| U83 | 65 | 90 | 6.5 | 38 | 2.1 |
| U84 | 65 | 90 | 7.5 | 40 | 8.1 |
| U85 | 95 | 90 | 6.5 | 40 | 4.8 |
| U86 | 95 | 90 | 7.5 | 38 | 5.5 |
| U87 | 95 | 110 | 6.5 | 38 | 4.7 |
| U88 | 95 | 110 | 7.5 | 40 | 6.6 |
| U89 | 65 | 110 | 6.5 | 40 | 5.4 |
| U90 | 65 | 110 | 7.5 | 38 | 1.8 |

### Results and Conclusion

Data comparison of the OD₆₀₀, the Fc-peptide fusion protein concentration and the DO of the three CP fermentation runs U79. U81 and U82 revealed that all three fermentations showed comparable results (Figure not shown). For example, the OD₆₀₀ increased continuously and followed similar courses in all three fermentations. The final OD₆₀₀ of the runs U79, U81 and U82 added up to 161, 199 and 190, respectively. The DO in the three cultures decreased in the first four hours to 30% where it was subsequently maintained by the control cascade. The end of the batch phase was reached in between 7.5 and 8.1 h which was signaled by an abrupt increase of the DO signal. After 17.1 h of fermentation time all three fermentation runs were induced by increasing the temperature to 39° C.

The influence of the four factors tested in a specific range on the maximum Fc-peptide fusion protein yield was evaluated mainly based on the calculated standard deviation (SD) of the CP results. The mean product concentration of the CP added up to 5.5±0.67 g/L.

Considering the standard deviation of the analytical method (about 15% for reducing SDS-PAGE analytics with Coomassie stain), no statistical correlation between the factor variance of induction OD₆₀₀, feed flow rate and pH to Fc-peptide fusion protein yield was detected given that the determined variability of product yield at EoF was within the range of standard deviation of the CP and the analytical method for almost all fermentations conducted during robustness testing. Thus, the process was considered robust regarding process parameter variations of induction OD₆₀₀, feed flow rate and pH tested in the investigated area.

However, a statistical correlation between Fc-peptide fusion protein yield at EoF and induction temperature was found (Table 20) in the course of the fermentation runs U83, U84, U88 and U90, where the Fc-peptide fusion protein yield at EoF was not within the range of the standard deviation. Both fermentations with an induction temperature of 38° C. (U83 and U90) showed the lowest EoF titers (2.1 and 1.8 g/L), while the fermentations U84 and U88 with an induction temperature of 40° C. showed the highest EoF titers of all fermentations (8.1 and 6.6 g/L).

In conclusion, the process regarding the parameter variation of induction OD₆₀₀, feed flow rate deviation and pH (OD_{Ind} 80±15, FFeed±10%. pH=7.0±0.5) was considered robust. However, the induction temperature showed a significant influence and was therefore identified as a critical process parameter which possibly influences the EoF yield within a deviation of 38±1° C. Therefore, it was concluded that the induction temperature should be closely monitored and controlled at 39° C. to ensure optimal Fc-peptide fusion protein production.

### Example 7: A Dissolved Oxygen (DO) Limitation of One Hour During Expression Phase was Considered as Feasible without any Negative Consequences on Cell Growth and Fc-Peptide Fusion Protein Production in 10 L Scale Fermentation Runs

Due to the implementation of the DO-control cascade with pressure, an additional fermentation was conducted to investigate possible effects of a potential DO-limitation during expression phase on Fc-peptide fusion protein production and cell growth.

For a detailed process description of the 10 L fermentation runs see “Materials and Methods”, above. Fermentation parameters and control loops are listed in Table 12 and in Table 13, respectively. The utilized glycerol containing KF1 pre-culture, culture and feed media are described in the “Materials and Methods” section in Tables 2, 3 and 6, respectively.

### Experimental Setup

Fermentation run U91 was conducted with induction OD₆₀₀ (OD_{Ind})=80.0±5, induction temperature (Tind)=39° C., growth rate (p)=0.1±0.001 l/h, KF1 culture and feed medium, Methionine concentration in KF1 feed solution (cMeth)=0 g/L, pressure DO-cascade, constant inlet air flow 1.5 vvm. The expression phase was prolonged until the pressure had reached its maximum value of 0.8 bar and the DO had dropped to 0%. Then, the process was monitored for one hour and additional samples were taken at the end of the fermentation for analytics.

### Results and Conclusion

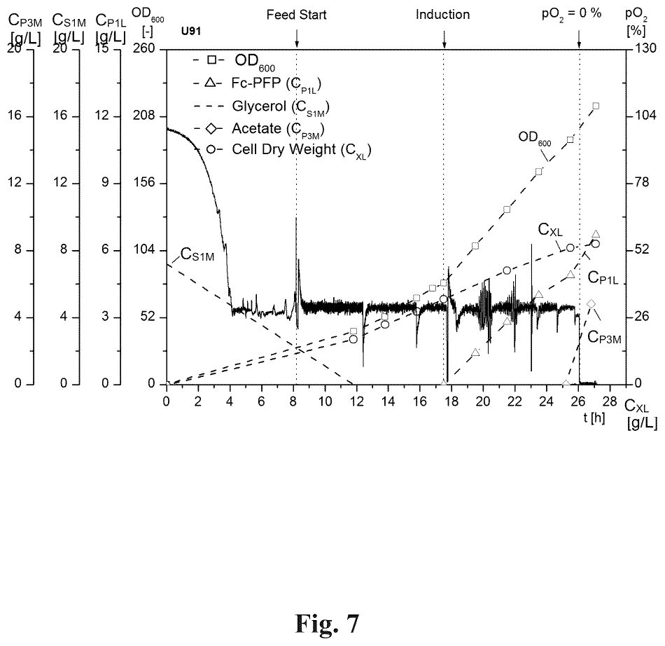

FIG. 7 shows the data of fermentation run U91. Throughout the process. OD₆₀₀, CDW and Fc-peptide fusion protein concentration increased continuously. The oxygen limitation during one hour did not have any effect on cell growth and product formation since both kept increasing after the DO had dropped to 0%. The final OD₆₀₀ value added up to 216 and the final Fc-peptide fusion protein concentration reached 6.7 g/L. A possible effect of the oxygen limitation was observed on the CDW. While CDW before DO-limitation added up to 53.0 g/L, it only increased to 54.5 g/L after one hour of limitation. Another remarkable effect was the increase of acetate in the medium. Prior to the oxygen limitation no acetate could be detected in the medium. After one hour of DO=0% the concentration of acetate had already reached 4.8 g/L.

Given that one hour of DO-limitation did not impair cell growth and product formation, and previous fermentations with the pressure control cascade at standard conditions were able to be processed for more than 8 h of expression time, the EoF criterion was defined for the final process as 8 h of expression. In the unlikely case of DO-limitation a maximum time for DO below 10% of one hour was considered as feasible without any negative consequences. Due to the accumulation of acetate in the medium, the reduction of CDW increase and the lack of experimental data beyond one hour of oxygen limitation, it was considered as not advisable to extend the process for more than one hour of DO-limitation.

### Example 8: Confirmation of Process Strategy in One Engineering Run at 10 L Scale

Having established a robust process with high Fc-peptide fusion protein titers at EoF, the final process strategy was conducted at 10 L scale to confirm the results of fermentation development.

For a detailed process description of the 10 L fermentation runs see “Materials and Methods”, above. Fermentation parameters and control loops are listed in Table 12 and in Table 13, respectively. The utilized glycerol containing KF1 pre-culture, culture and feed media are described in the “Materials and Methods” section in Tables 2, 3 and 6, respectively.

### Experimental Setup

Briefly, fermentation run U93 was conducted with induction OD₆₀₀ (OD_{Ind})=80.0±5 (82.5±2.5), induction temperature (Tind)=39° C., growth rate (μ)=0.1±0.001 l/h, KF1 culture and feed medium, Methionine concentration in feed solution KF1 (cMeth)=0 g/L, expression time (texpr)=8 h, pressure DO-cascade, and constant inlet air flow 1.5 vvm.

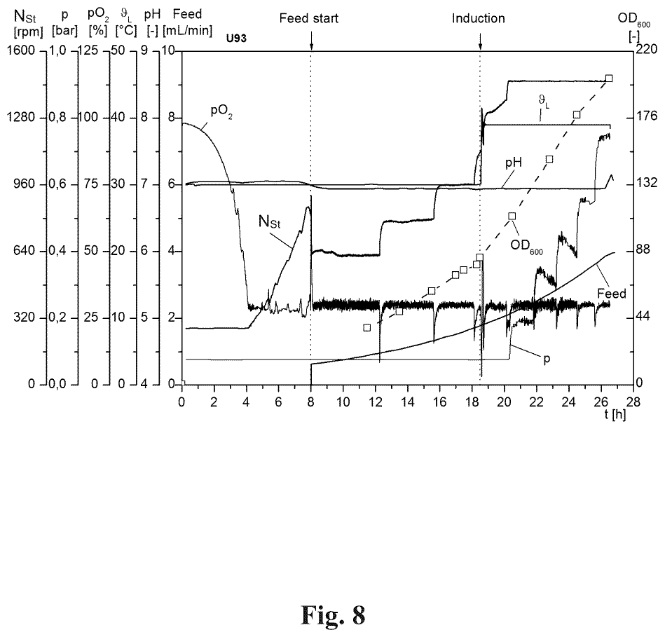

FIG. 8 shows the online data from fermentation run U93. The stirrer speed started rising after the DO had reached 30% to keep it at its set point. After 8 h the end of the batch phase was signaled by a sharp rise of DO-signal which automatically triggered the defined exponential feed profile. The induction of the culture was conducted after 18.5 h by increasing/ramping the temperature from 30° C. to 39° C., as the OD₆₀₀ had reached the defined induction range of 80-85. After about 20.5 h the stirrer reached its maximum speed of 1.500 rpm and thus, the pressure, in the reactor was increased from 0.0 to 0.75 bar at EoF (maximum possible pressure 0.8 bar) to maintain the DO at 30%. After the previously defined 8 h of expression, the fermentation was terminated, and the fermentation broth was subsequently cooled to 20° C.

### Results and Conclusion

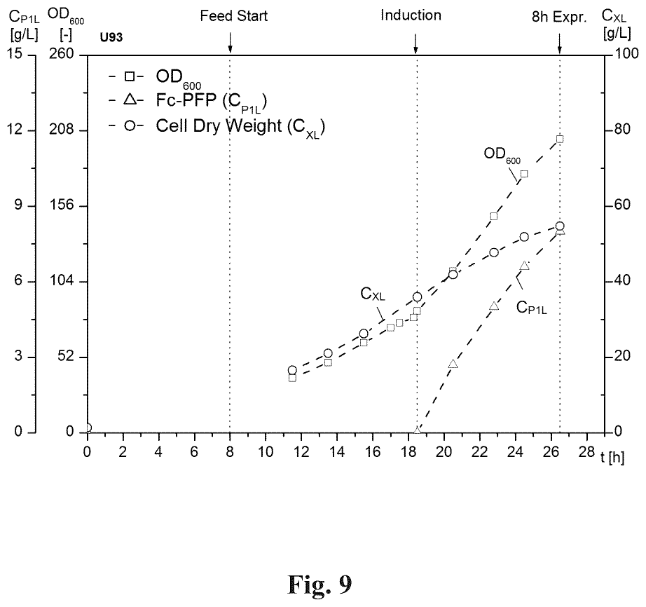

The offline data from sample analytics for the pilot run U93 is shown in FIG. 9. The OD₆₀₀ of the fermentation increased in the first 18.5 h continuously to a value of 84. After induction, the temperature was maintained at 39° C. during the following 8 h of expression which probably caused the growth to rise slightly. The final OD₆₀₀ after 26.5 h of fermentation time added up to 202. The cell dry weight (CDW) grew constantly throughout the fermentation and added up to 54.7 g/L at the end of the fermentation. After induction, the Fc-peptide fusion protein concentration increased in 8 h of expression continuously to a final value of 8.0 g/L.

Given that the cells showed continuous growth throughout the process, that the Fc-peptide fusion protein concentration was among the highest ever achieved with this process strategy and that 8 h of expression could be performed without reaching the pressure cascade maximum and thus causing a decrease of DO, the pilot run and the developed Fc-peptide fusion protein manufacturing process was confirmed and was considered successful.

Some of the development phase findings which have been derived from Examples 1 to & are summarized in Tables 21 and 22.

TABLE 21
First development phase results

| Unit | Investigated Parameter/ | |
|---|---|---|
| Operation | Description | Results/Decision |
| DoE | Variation of OD_{Ind}, μ, T_{Ind} and | High OD_{Ind} and low T_{Ind} had positive |
| cultivations | cₘₑₜₕ and identification of impact | influence on Fc-peptide fusion protein |
| | on Fc-peptide fusion protein | production; cₘₑₜₕ was irrelevant. High |
| | yield | μ showed high productivity. OD_{Ind} = |
| | | 80, μ = 0.2 1/h, T_{Ind} = 39° C. |
| | | was implemented for process strategy. |
| Post-DoE | Impact of kanamycin | No impact of kanamycin on process. |
| experiments | Impact of dissolved oxygen (DO) | Lower DO set point had minor |
| | set point | negative effect on growth and Fc- |
| | | peptide fusion protein production. |

TABLE 22
Second development phase results

| Unit | Investigated Parameter/ | |
|---|---|---|
| Operation | Description | Results/Decision |
| Scale up | Scale up to 10 L | 10 L fermentation data comparable to |
| | | results in 1 L scale. |
| | Comparison of sterilization | Filter sterilization of feed solution had |
| | methods for feed solution | no impact on process - change to filter |
| | | sterilization. |
| | DO-control cascade with tank | Change to μ = 0.1 1/h - DO-control |
| | pressure | cascade successfully implemented with |
| | | 8 h of expression. |
| Robustness | Variation of OD_{Ind}, T_{Ind}, pH | Process is robust regarding OD_{Ind}, pH |
| | and F_{Feed} | and F_{Feed}. T_{Ind} however was identified |
| | | as critical parameter. |
| | DO limitation during expression | DO = 0% for 1 h did not have negative |
| | | effect on growth and Fc-peptide fusion |
| | | protein production. |

In conclusion, the induction temperature showed a significant effect on Fc-peptide fusion protein production and a low induction temperature of about 38° C. to 40° C., preferably of 39° C. was identified as most suitable for obtaining a high Fc-peptide fusion protein production yield, whereas the process was robust regarding process parameter variations of induction OD₆₀₀, feed flow rate and pH. Nevertheless, an induction OD₆₀₀ of at least 30 was identified as 10 suitable for obtaining a high product yield, wherein a higher induction OD₆₀₀ of for example 80 even increased Fc-peptide fusion protein yield. Furthermore, the growth rate had no impact on product yield, but only on the productivity which was optimal with a growth rate of 0.2 l/h. In addition, a low DO set point and even a DO of 0% for about 1 hour had no negative effect on cell growth and Fc-peptide fusion protein production.

What is claimed is:

1. A process suitable for the large scale recombinant production of an Fc-peptide fusion protein in recombinant prokaryotic host cells, wherein the process comprises a batch phase, a feeding phase before induction, and a temperature induction phase, wherein the process comprises the steps of:
a) culturing the recombinant prokaryotic host cells during the batch phase and the feeding phase at a first cultivation temperature of about 27° C. to about 35° C. in a culture medium, wherein the recombinant prokaryotic host cells comprise a nucleic acid encoding the Fc-peptide fusion protein which is operably linked to a temperature inducible promoter;
b) starting the temperature induction phase at an OD₆₀₀ of >30 through a temperature shift towards a second cultivation temperature of about 38° C. to about 40° C., thereby inducing the expression of the Fc-peptide fusion protein; and
c) maintaining the second cultivation temperature for at least 4 hours.

2. The process according to claim 1, wherein the recombinant prokaryotic host cells are Escherichia coli cells.

3. The process according to claim 2, wherein the Escherichia coli cells are Escherichia coli BL21 cells.

4. The process according to claim 1, wherein the temperature inducible promoter is a lambda PR promoter which is regulated by a temperature sensitive repressor.

5. The process according to claim 4, wherein the temperature sensitive repressor is lambda cI857 repressor.

6. The process according to claim 1, wherein the first cultivation temperature is 30° C.±1° C.

7. The process according to claim 1, wherein culturing the recombinant prokaryotic host cells according to step a) occurs in the presence of glycerol as a carbon source.

8. The process according to claim 1, wherein:
1) the culture medium used in the feeding phase does not comprise methionine,
2) the culture medium used in the batch phase does not comprise an antibiotic,
3) the culture medium used in the feeding phase does not comprise an antibiotic,
4) or any combination thereof.

9. The process according to claim 1, wherein culturing the recombinant prokaryotic host cells according to step a) occurs at a growth rate of 0.05 to 0.3 doublings per hour.

10. The process according to claim 9, wherein the growth rate is 0.1 doublings per hour.

11. The process according to claim 1, wherein starting the temperature induction phase according to step b) occurs at an OD₆₀₀ of 80±5, and the second cultivation temperature is 39° C.

12. The process according to claim 1, wherein starting the temperature induction phase according to step b) occurs at an OD₆₀₀ of 82.5±2.5, and the second cultivation temperature is 39° C.

13. The process according to claim 1, wherein the first cultivation temperature is 30° C., and the second cultivation temperature is 39° C.

14. The process according to claim 1, wherein the second cultivation temperature is maintained in step c) for 5 to 10 hours.

15. The process according to claim 1, wherein the second cultivation temperature is maintained in step c) for 6 to 8 hours.

16. The process according to claim 1, wherein the second cultivation temperature is maintained in step c) for 8 hours.

17. The process according to claim 1, wherein the process is performed in a bioreactor, wherein a dissolved oxygen (DO) concentration is maintained.

18. The process according to claim 17, wherein the dissolved oxygen (DO) concentration is maintained above 8%.

19. The process according to claim 17, wherein the dissolved oxygen (DO) concentration is maintained above 10%.

20. The process according to claim 17, wherein the dissolved oxygen (DO) concentration is maintained above 30%.

21. The process according to claim 17, wherein the dissolved oxygen (DO) concentration is maintained through control of a bioreactor pressure.

22. The process according to claim 21, wherein the bioreactor pressure is increased from 0.0 to 0.8 bar(g).

23. The process according to claim 1, wherein the Fc-peptide fusion protein is expressed in an insoluble or a limited soluble form and is accumulated in inclusion bodies.

24. The process according to claim 1, wherein the process further comprises a step of:
d) harvesting the Fc-peptide fusion protein from the recombinant prokaryotic host cells or from the cell culture medium.

25. The process according to claim 24, wherein step d) further comprises a step of isolating the Fc-peptide fusion protein containing inclusion bodies by a sedimentation step.

26. The process according to claim 25, wherein the sedimentation step is a centrifugation step.

27. The process according to claim 24, wherein step d) further comprises a step of refolding the Fc-peptide fusion protein.

28. The process according to claim 24, wherein step d) further comprises a step of purifying the Fc-peptide fusion protein.

29. The process according to claim 28, wherein the step of purifying the Fc-peptide fusion protein is performed by a method comprising the following steps in the following order:
i) performing an affinity capture chromatography in bind-elute mode:
ii) performing a mixed-mode chromatography in bind-elute mode;
iii) performing a cation exchange chromatography in bind-elute mode; and
iv) performing an ultrafiltration/diafiltration.

30. The process according to claim 29, wherein the chromatographic step i), ii), or iii) is performed with one or more washing steps.

31. The process according to claim 1, wherein the Fc-peptide fusion protein is a monomer, a dimer, or a polymer.

32. The process according to claim 1, wherein the Fc-peptide fusion protein is a dimer.

33. The process according to claim 1, wherein the Fc-peptide fusion protein is a receptor agonist.

34. The process according to claim 33, wherein the receptor agonist is a thrombopoietin mimetic.

35. The process according to claim 34, wherein the thrombopoietin mimetic is romiplostim.

36. A method of manufacturing a pharmaceutical composition comprising an Fc-peptide fusion protein, wherein the method comprises a batch phase, a feeding phase before induction, and a temperature induction phase, wherein the method comprises the steps of:
a) culturing recombinant prokaryotic host cells during the batch phase and the feeding phase at a first cultivation temperature of about 27° C. to about 35° C. in a culture medium, wherein the recombinant prokaryotic host cells comprise a nucleic acid encoding the Fc-peptide fusion protein which is operably linked to a temperature inducible promoter:
b) starting the temperature induction phase at an OD₆₀₀ of >30 through a temperature shift towards a second cultivation temperature of about 38° C. to about 40° C., thereby inducing the expression of the Fc-peptide fusion protein;
c) maintaining the second cultivation temperature for at least 4 hours;
d) harvesting the Fc-peptide fusion protein from the recombinant prokaryotic host cells or from the cell culture medium; and
e) formulating the Fe-peptide fusion protein with a pharmaceutically acceptable carrier or buffer.

37. The method of claim 36, wherein the method further comprises filling the pharmaceutical composition in a pharmaceutical container.

38. The method of claim 36, wherein the method further comprises lyophilizing the pharmaceutical composition.

39. The method of claim 36, wherein the Fc-peptide fusion protein is romiplostim.

40. The method of claim 36, wherein the pharmaceutical composition further comprises L-histidine, mannitol, polysorbate 20, sucrose, and HCl.

41. The method of claim 36, wherein the pharmaceutical composition is in the form of a sterile and preservative-free white powder.

42. The method of claim 36, wherein the pharmaceutical composition is contained in a pharmaceutical container.

43. The method of claim 42, wherein the pharmaceutical container is a single-dose vial.

44. The method of claim 43, wherein the single-dose vial comprises 230 μg romiplostim, 0.7 mg L-histidine, 18 mg mannitol, 0.02 mg polysorbate 20, 9 mg sucrose, and sufficient HCl to adjust the pH to a target of 5.0.

45. The method of claim 43, wherein the single-dose vial comprises 375 μg romiplostim, 1.2 mg L-histidine, 30 mg mannitol, 0.03 mg polysorbate 20, 15 mg sucrose, and sufficient HCl to adjust the pH to a target of 5.0.

46. The method of claim 43, wherein the single-dose vial comprises 625 μg romiplostim, 1.9 mg L-histidine, 50 mg mannitol, 0.05 mg polysorbate 20, 25 mg sucrose, and sufficient HCl to adjust the pH to a target of 5.0.
